(19) United States
(12) Patent Application Publication
(10) Pub. No.: US 2008/0228040 A1
(43) Pub. Date: Sep. 18, 2008

(54) International medical expert diagnosis

(76) Inventor: Arthur Solomon Thompson, Reston, VA (US)

Correspondence Address: Arthur Solomon Thompson, 11904 Winterthur Lane, Reston, VA, 20191, US

(21) Appl. No.: 11/723,138

(22) Filed: Mar. 16, 2007

(51) Int. Cl. A61B 5/00

(52) U.S. Cl. 600/300

(57) ABSTRACT

The delivery of services internationally has been stressful for consumers for several decades based on a lack of physical resources and increasing needs of individuals. Health care service delivery has been under close scrutiny since most countries cannot define viable alternatives to care for it population. Optimized Intelligent Search technology affords an opportunity to provide expert system caliber services on PDA and other computer platforms internationally based on leveraging unparalleled search capability, leveraging of decades of past history in seconds and provides optimized processes implementation for efficient use of resources.

Use of intelligent agent technology will provide a leveraging of existing resource to a vast number of people. Expert systems in coalition with the internet technology will provide search resources internationally to make health research accessible with the possibility of identifying agencies that have resources to help. The patent discusses the use of leveraging a frequency map in the diagnosis processes since the body is a frequency spectrum. Mapping frequencies to body components allow the enumeration of the power set of frequencies which are known behaviors since we know the base 200 body component frequencies. (2̂200) frequencies are known which are potential treatment strategies. Frequency entrainment of application of a restoring frequency to a distorted signal will provide relief in a number of health venues.

This application claims the benefit of the filing date of Provisional application US 60/783,036 Filed in Mar. 17, 2006

### REFERENCE

Computerized medical advice systsem and method including Meta function U.S. Pat. Nos. 5,711,297, 5,012,411

### NO FEDERALLY SPONSORED RESEARCH AND DEVELOPMENT

Drawing attached

1. Background of the Invention

This specification contains no new information since the annotations were derived from the drawing document per 37 CFR 1.125 (b).

PDA facilitates the fundamental health care delivery issue which involves access to service. The current implementation of this expert system technology is available to participant who has a PDA or cellular phone access or other computerized device.

2. Prior Art: The Objects and Advantages of the Invention

Traditional Medical Diagnostic systems take user input in the form of a collection of symptoms in an effort to speculate a probable cause or diagnosis. The subject Optimized intelligent Architecture leverages unparalleled search, past history and processing power to provide an improved reliability in predicting the progression of human illness. Homeostasis is defined in terms of metrics which define good health. There are two hundred body parts including blood, urine and vital signs which are incorporated in the diagnostic strategy.

Specifically we are analyzing a business object which will be offered as an internet service internationally. The key aspect of this analysis is simplicity and scalability of medical services offerings to a multi-lingual international community. The target platform is the Business phone which serves as an internet services conduit to a number of diversified markets. PDA requirements are that it can read a web page and support configuration to support a menu system of 200 human components complementing a diagnostic system. Each of the 200 human body components are either pristine or in symptom state. A Medical Diagnostic system of good caliber is ubiquitous especially one that leverages past medical history of patient for present diagnosis in a few seconds. An Expert System caliber shell device on a PDA is also ubiquitous since it accommodates a number of different expert system applications on the same interface. The pattern search strategy appears to be many times more powerful than most if not all of the convention search strategies that are currently mainstreamed. Low priced expert systems of this caliber will also make it affordable and easily incorporated in a number of different languages and collaboration settings. Artificial Intelligence is underscored by search (intelligent) and search engine technology will probably dominate many computer driven initiatives, hence the search technology leveraged in this endeavor will be the commodity which redefines the foregoing expert system implementation.

### DESCRIPTION OF DRAWINGS

FIG. 1. Data View or interface where the user of the expert system must define a collection of basis objects which span the problem space. It is assumed that a linear combination of any set of these basis objects will correspond with any instantiation in the domain. Typical collection of basis vectors for Expert Systems Medical, Trouble Shooting, Enterprise Resource, Chess, Genome are derived by experts or system professionals

FIG. 2. Interface defining Expert Shell reusable container in the expert system (minus the basis vectors) which facilitates the search for the correct set of states which identifies a set of aberrant conditions or in the case of symptoms a medical diagnosis. An expert system answers the questions the customer asks.

FIG. 3. Piece Removal facilitates Search Problem by removing objects from game tree (e.g. capture in chess game or pattern reduction strategy. It appears that piece reduction in a games venue is one of the unparalleled search strategy mainstreamed.

FIG. 4. Set Complement determines all missing data from the target set or basis set of objects. It is a set operation which keeps track of missing information and object location. In performing diagnosis or system analysis on a system we assume the initial state of homeostasis defines a well working collection of components. As system inconsistencies and flaws arise, the diagnosis process keeps track of all the original start states of the system, collects all the system aberrations or symptoms and identifies the set of objects which needs to be restored to make the system complete. The forte of the diagnosis process is the ability of keeping track of trillions of trillion of system states leveraging a search process that is fundamentally derived from the chess piece capture process.

FIG. 5 Some keys pressed down indicating symptoms. This is what is known as an analog of a key being pressed on a piano system and a symptom being realized in a human body system.

FIG. 6 Indicates keys to restore to un-pressed state. Fundamentally an analog process model relating a healing process and releasing a collection of pressed piano notes.

FIG. 7 Symptom graph or a group of symptoms is a diagnosis. Depiction of the graph illustrates the total collection of states an illness can take on. This could be represented as sickness diagnosis space.

FIG. 8 Model of frequency break-down of the human body. As a spectrum is should be possible to identify or communicate with by components once frequency ranges are known.

FIG. 9 Time, frequency, visit count, and body part is an instantiation of a medical scenario which should be recorded and maintained has past medical history.

FIG. 10 Each body part has a corresponding frequency and visit count recording past history. Model of a communication strategy between frequency and body part.

FIG. a10A Data flow diagram shows piano analogy of medical expert system design. Specifically the instrument has 200 keys or frequencies which map to 200 body parts. The body communication key communications conduit is frequency since each body component has a unique frequency.

User Steps;
User experiences symptoms from some unknown medical condition
User takes his PDA device and presses medical history which provides several decades of past medical history.
User accesses 200 body component menus and carefully selects the components that have associated symptoms.
As the user selects symptoms the system questions the participant with other likely components from the body system that may have contributed to the illness. These questions originate from past medical history instantiated by system visit count or the propensity to go in medical system states based on past behavior. System calibrates based on the symptoms selected and provides a diagnosis.

Treatment
System leverages frequency information obtained during the diagnosis phase and uses ultra-sound as a means of correcting or entraining aberrant frequency readings.
Oscillator vibrates until weak symptom signal from the body component is corrected
Record date of each visit count for leveraging past history
Oscillator Scanner—Bad frequency corrected by entrainment

FIG. 11 Remembers process steps by tracking past history facilitate the maximum opportunity for a practitioner to identify the cause of an illness.

FIG. 12 Specifying a set of basis frequencies for body components will provide a very scalable treatment strategy leveraging the power set of the basis set as potential treatments or a linear combination of the basis set. Each procedure maps to a unique frequency.

FIG. 13 International Medical Expert Diagnostics or an entity which will provide medical diagnosis and patient assistance nation wide.

FIG. 14 Maps objects to bit domain to facilitate system modeling.

FIG. 15 Shows epochs of objects to simulate medical scenarios

FIG. 16 Collection of bits is objects a modeling technique which scales tremendously facilitating extreme search and leveraging of past history.

FIG. 17 Device diagnosing symptoms and scanning frequencies

FIG. 18 Host human with symptoms

FIG. 19 Depicts sick condition or unorganized and out of phase vibration condition.

FIG. 20 Frequency Entrainments—corrects bad signal. The appropriate signal will cause an aberrant frequency condition to be rectified.

FIG. 21 Entrain signal until rectified may facilitate a cure. Adding a more dominant frequency to a waveform may restore it to its' proper vibration state.

FIG. 22 Historical visit count is medical history leveraged with present symptoms of body parts and visit count (past history). Body components and frequency distribution showing system visit count.

FIG. 23 Analog for human body frequency distribution showing each component can be communicated by a unique frequency.

There are a variety of data flow processes drawing which outline how information is passed through each phase of the process. Some of the drawings actually depict the as built system illustrating the GUI interface. See drawing p p 17, p 19, p 23, and p 42

FIG. 24 Maps any collection to a set of critical paths based on captures state architecture.

FIG. 25 Model leverage past history capturing linear and non-linear pattern presentation.

FIG. 26 Linear Illness String is processed in a graph which enumerates all states of a set of three.

FIG. 27 An enumeration scheme showing a non-linear transition or skipping graph level may be viewed as a non-linear transition.

FIG. 28 Depiction of a collection of body components which are in a state of homeostasis or good health. No symptoms encountered

FIG. 29 A collection of a set of body parts that have symptoms of some nature.

FIG. 30. Shows a past history of symptoms and some present states without symptoms.

FIG. 31 Shows a present state of an undiagnosed condition

FIG. 32 Shows a graph representing a general system problem solver leveraging intelligent search, past history, processing power and classification.

FIG. 33 Makes reference to a language which will be derived from the symptom-diagnostic engine. The symptom diagnosis engine associates a group of symptoms which are represented in a numerical format to facilitate object association.

FIG. 34 Graphic of the international Medical Expert Diagnosis and basic template for 60 other Expert System Designs discussed later.

FIG. 35 Template or example of International Medical Expert Diagnosis.

FIG. 36. Electronic Medical Record on a Personal Data Assistant (PDA) providing for Expert System functionality.

FIG. 37 Electronic Medical Data on a Personal Data Assistant facilitates data access anywhere.

FIG. 38 Graphic of system question interface in each body system helps to narrow down actual cause of symptoms

FIG. 39 Graph showing capture and optimal path modeling. The capture graph is the heart of the piece removal or pattern reduction and search finding strategy.

FIG. 40 Illustration of a connectivity graph associates information about gene and other body component mappings.

FIG. 41 Biological Transfer Function provides a process flow model on illness symptoms and a strategy to track and reverse aberrant trends.

FIG. 42 Strategy to leverage past history for games dog fight by recording the number of times a fighter jet reused an object from a basis collection of alternatives.

FIG. 43 Leverage all available past aircraft fighter history in order to make a tactical decision in a few seconds.

FIG. 44 List all patterns and all past history for fighter dog fight to enhance their ability to take evasive maneuvers.

FIG. 45 Weight distribution and Evaluation function facilitating decision making

FIG. 46 Decision in chess game may be based on piece weight and by system visit count. This model does not emphasize piece position although it can incorporate this parameter.

FIG. 47 Software robot pattern recognizer, leveraging past history to answer questions and provide services

FIG. 48 Business Model for game fighter jet summarizing variables and constraints.

FIG. 49 Optimized Intelligent Search identifies business collections for the purposes of documenting characteristics and behaviors.

FIG. 50 Enterprise Resource Planning Business Model and problem formulation. Provide a searchable knowledge base which leverages past history to answer business reconciliation issues.

FIG. 51 Optimized Intelligent Search Expert Systems and Search Applications. Collection of over 60 applications which can be formulated and configured in a very extremely efficient and robust process which may facilitate formal methods programming control.

FIG. 52 Optimized Intelligent Search Expert Systems and Search Applications 01-15

FIG. 53 Optimized Intelligent Search Expert Systems and Search Applications 16-30

FIG. 54 Optimized Intelligent Search Expert Systems and Search Applications 31-45

FIG. 55 Optimized Intelligent Search Expert Systems and Search Applications 46-60

Intelligent Search Technology
1. Optimal Intelligent Search (OIS) is a powerful search strategy that was designed for application software but targeted at web enabled expert systems applications. As objects are removed from the search stack the search space is divided in half. This architecture is intended to allow high end expert systems to run on a web page leveraging minimal code. OIS exploits the search heuristic of a graph by traversing the height of the graph or levels based on Boolean lattice architecture. Leveraging Past History real-time (within seconds) provides the ability to make critical decisions in a medical context, financial strategy and in general production systems which support very complex procedures. OIS maps a collection to a set of unit basis vectors, recording the visit count to these systems as a means of capturing past history in addition traversing an intractably large search space in linear time.
2. OIS is projected to enable Nano Control Structures providing for pattern recognition, autonomous or group heuristics governing controlling large farms of Nano particles. Because of the tiny size of these particles, Nano objects will provide a unique opportunity to explore the human body and detect a variety of aberrant conditions. Nano Control Structure may provide the most accurate and comprehensive feedback mechanism for Medical Expert Diagnostic Systems. It is also speculated that the level of control may go beyond detection, into an actual behavior driven interactive response system. Full realization of Nano control may involve performing operations which are impossible leveraging current technology, like surgery in currently inaccessible areas of the body.

Optimized Intelligent Search
Search
Leveraging Past History
Processing Power
Applications
Medical Expert System PDA
Medical Expert System Web Apps
Applications

Expert System

Briefly an Expert System should “Answer the question the Customer Asks”. Pertaining to Medical Diagnostics, the Objective of a Medical Diagnostic Expert System is to diagnose any probable collection of Symptoms. The Medical Oracle gives an accurate diagnosis (consistent with a given set of symptoms) based on a knowledge base of previous similar case histories. The complement process piece reduction procedure, and automatic patient case history clustering provide strategies for identifying cure alternatives. The following will demonstrate expert search, classification and evaluation and leveraging past history for future medical diagnosis.

Pattern recognition (also known as classification) is a discipline in computer science characterized by “capturing raw data and taking an action based on the category of the data. It uses methods from statistics, machine learning and other areas.” Specifically we are interested in recognizing patterns in Diagnosis and treatment analysis.
The basic technology is a form of Artificial Intelligence that ‘gives computers the ability to recognize patterns the way humans can; specifically the ability to leverage past history to make present decisions.
“Machine learning refers to a system capable of the “autonomous acquisition and integration of knowledge”. This capacity to learn from experience, analytical observation, and other means, results in a system that can continuously self-improve and thereby offer increased efficiency and effectiveness.

Scalability & Search Ability
This search strategy instantaneously keeps track of more than 2̂100 symptom ridden cases.
An implement able strategy of modeling and managing conditions down to the atomic level is difficult to imagine outside this context. The 32 Trillion cells in the body can be characterized by a collection of 45 bits and the corresponding power set (2̂45˜32 trillion). The classification and symptom clustering rules are part of the infrastructure. Based on the aforementioned, the Medical Oracle will meet requirements for human genome modeling and testing and will ultimately provide the search and evaluation architecture of choice
The System Solution Strategy invariably involves creating a linked list or collection of Basis vectors (span search space e.g. . . . x,y,z) then apply a search strategy to identify a collection that meets a set of conditions.

Expert System

Briefly an Expert System should “Answer the question the Customer Asks”. Pertaining to Medical Diagnostics, the Objective of a Medical Diagnostic Expert System is to diagnose any probable collection of Symptoms. The Medical Oracle gives an accurate diagnosis (consistent with a given set of symptoms) based on a knowledge base of previous similar case histories. The complement process piece reduction procedure, and automatic patient case history clustering provide strategies for identifying cure alternatives. The following will demonstrate expert search, classification and evaluation and leveraging past history for future medical diagnosis.

Pattern recognition (also known as classification) is a discipline in computer science characterized by “capturing raw data and taking an action based on the category of the data. It uses methods from statistics, machine learning and other areas.” Specifically we are interested in recognizing patterns in Diagnosis and treatment analysis.
The basic technology is a form of Artificial Intelligence that gives computers the ability to recognize patterns the way humans can; specifically the ability to leverage past history to make present decisions.
“Machine learning refers to a system capable of the “autonomous acquisition and integration of knowledge”. This capacity to learn from experience, analytical observation, and other means, results in a system that can continuously self-improve and thereby offer increased efficiency and effectiveness.

Scalability & Search Ability
This search strategy instantaneously keeps track of more than 2̂100 symptom ridden cases.
An implement able strategy of modeling and managing conditions down to the atomic level is difficult to imagine outside this context. The 32 Trillion cells in the body can be characterized by a collection of 45 bits and the corresponding power set (2̂45˜32 trillion). The classification and symptom clustering rules are part of the infrastructure. Based on the aforementioned, the Medical Oracle will meet requirements for human genome modeling and testing and will ultimately provide the search and evaluation architecture of choice
The System Solution Strategy invariably involves creating a linked list or collection of Basis vectors (span search space e.g. . . . x,y,z) then apply a search strategy to identify a collection that meets a set of conditions.

Expert System
Briefly an Expert System should “Answer the question the Customer Asks”. Pertaining to Medical Diagnostics, the Objective of a Medical Diagnostic Expert System is to diagnose any probable collection of Symptoms. The Medical Oracle gives an accurate diagnosis (consistent with a given set of symptoms) based on a knowledge base of previous similar case histories. The complement process piece reduction procedure, and automatic patient case history clustering provide strategies for identifying cure alternatives. The following will demonstrate expert search, classification and evaluation and leveraging past history for future medical diagnosis.

Pattern recognition (also known as classification) is a discipline in computer science characterized by “capturing raw data and taking an action based on the category of the data. It uses methods from statistics, machine learning and other areas.” Specifically we are interested in recognizing patterns in Diagnosis and treatment analysis.
The basic technology is a form of Artificial Intelligence that ‘gives computers the ability to recognize patterns the way humans can; specifically the ability to leverage past history to make present decisions.
“Machine learning refers to a system capable of the “autonomous acquisition and integration of knowledge”. This capacity to learn from experience, analytical observation, and other means, results in a system that can continuously self-improve and thereby offer increased efficiency and effectiveness.

Scalability & Search Ability
This search strategy instantaneously keeps track of more than 2̂100 symptom ridden cases.
An implement able strategy of modeling and managing conditions down to the atomic level is difficult to imagine outside this context. The 32 Trillion cells in the body can be characterized by a collection of 45 bits and the corresponding power set (2̂45˜32 trillion). The classification and symptom clustering rules are part of the infrastructure. Based on the aforementioned, the Medical Oracle will meet requirements for human genome modeling and testing and will ultimately provide the search and evaluation architecture of choice
The System Solution Strategy invariably involves creating a linked list or collection of Basis vectors (span search space e.g. x,y,z) then apply a search strategy to identify a collection that meets a set of conditions.

Explanation of the Chess Space Search Problem
The chess game is the classic example of an intractable search problem which was achievable based on removing pieces from the game board to eliminate a seemly inexhaustible pattern space. Piece remove appears to be a very robust and effective way of reducing pattern space and is central to the search, leveraging past history and processing power defined herein.
The intractable large search space was used to model very complex systems in medicine, business, and information science and has proven to provide unparalleled search, memory of history and processing power primarily in the context of expert system and software robot design.
The capture states of a chess game was utilized to leverage a finite set of basis vectors which was removed by defining medical symptoms or removed to satisfy a search heuristic (process of elimination). The capture states in chess actually embody the behavior of the game since the piece moves to capture are trivial and are to intractable to remember. In the context of achieving capture states we can remember past episode and leverage this past history in seconds.
Expert System in layman's terms is an architecture which facilitates finding the best path or process by associating and optimizing all available data.
There may be only a few architectures which Optimally leverages many types of datasets.
There should be two types of sets:
Universal set (all available data)
Domain set (specialized information

Combinatorial Analysis of Chess

Chess Search and Capture Model Design

The entire tree for all possible chess moves, the total number of board positions is about 1,000,000,000,000,000,000,000,000, 000,000,000,000,000,000,000,000,000,000,000,000,000,000, 000,000,000,000,000,000,000,000,000,000,000,000,000,000, 000,000,000,000, or 10̂120,

It would take over 10̂60 centuries to generate the entire search tree at each successor at ⅓ of a Nano Second.

Optimize Intelligent Search leverages the search power of base 2 which can find in 400 finite steps any point in the 10̂120 search space˜2̂400. This approach provides the optimal path to each point or set of systems in the search domain. None of the games are reusable with the chess architecture however just about all games using the capture model are reusable.

A microcomputer can process 400 bits in a few seconds.

Leveraging piece removal or piece capture provides for an unparalleled search strategy eliminating just about all of the 10̂120 patterns by removing 32 chess pieces. Removing a piece is like dividing the search space in half 222222222222222222222222222222222222 . . . 2̂400

Expert Systems Applications

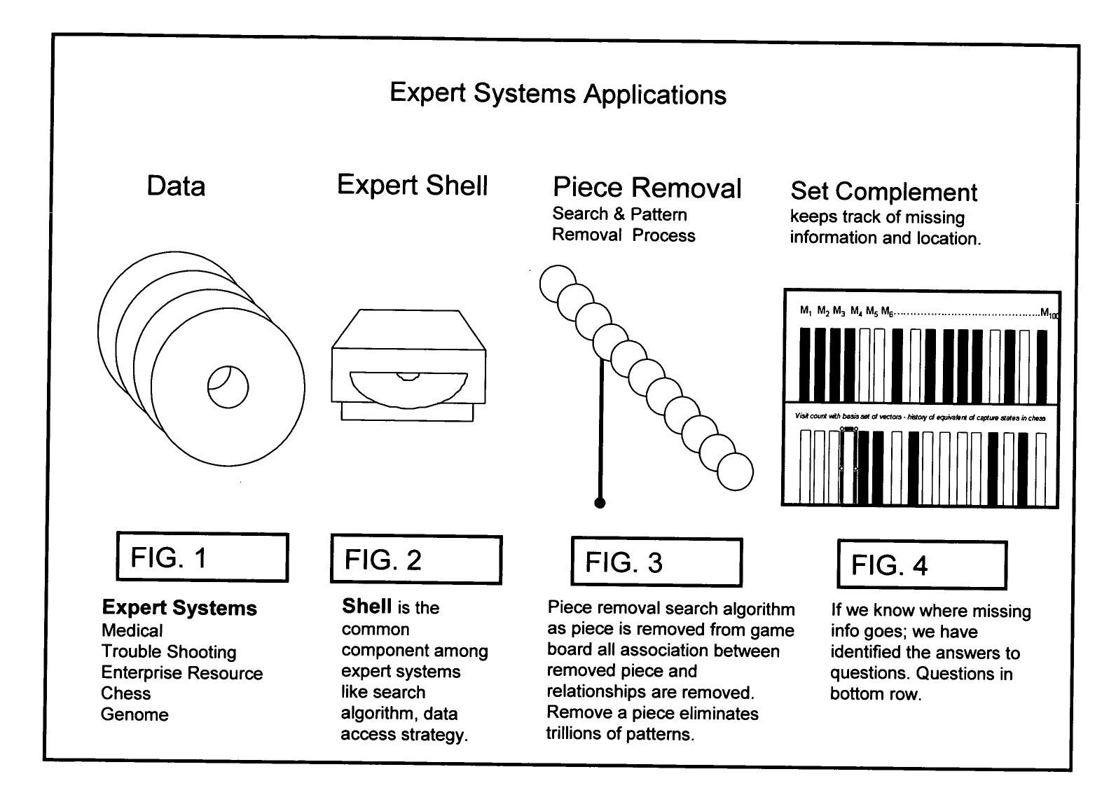

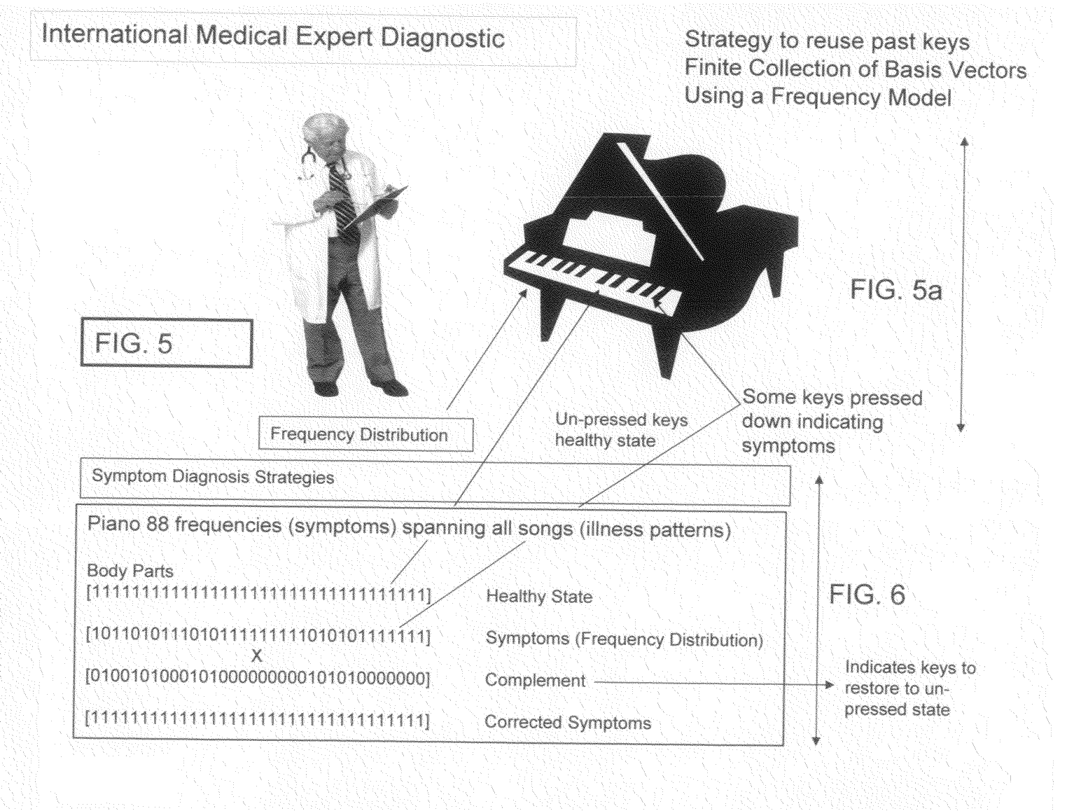

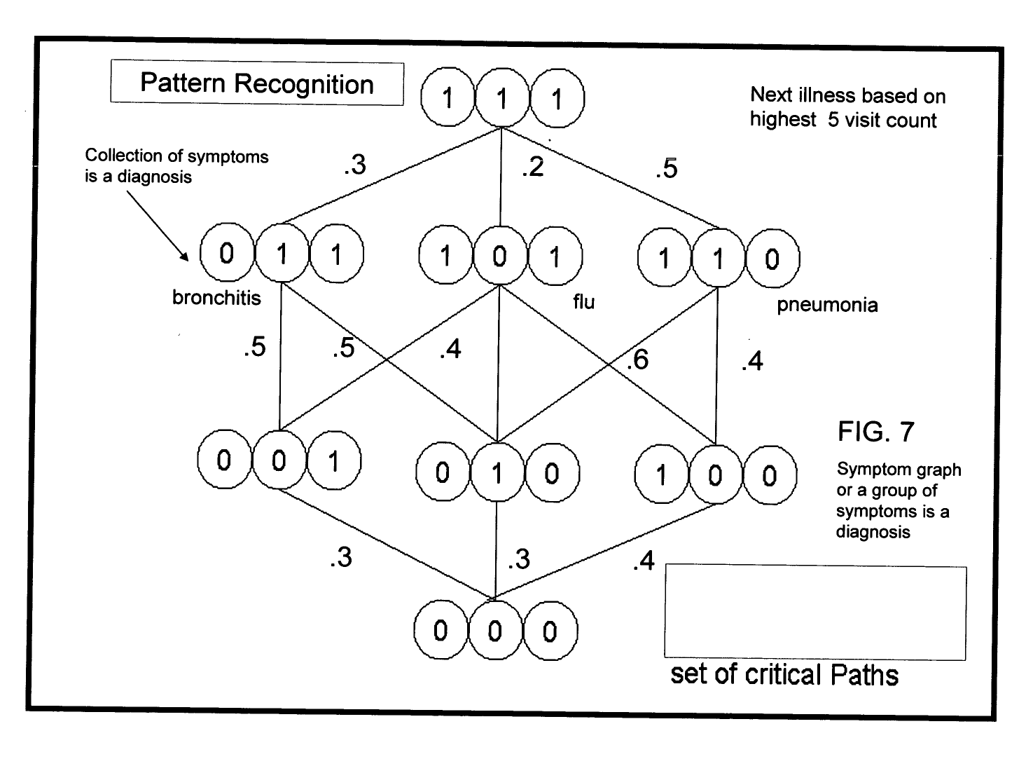

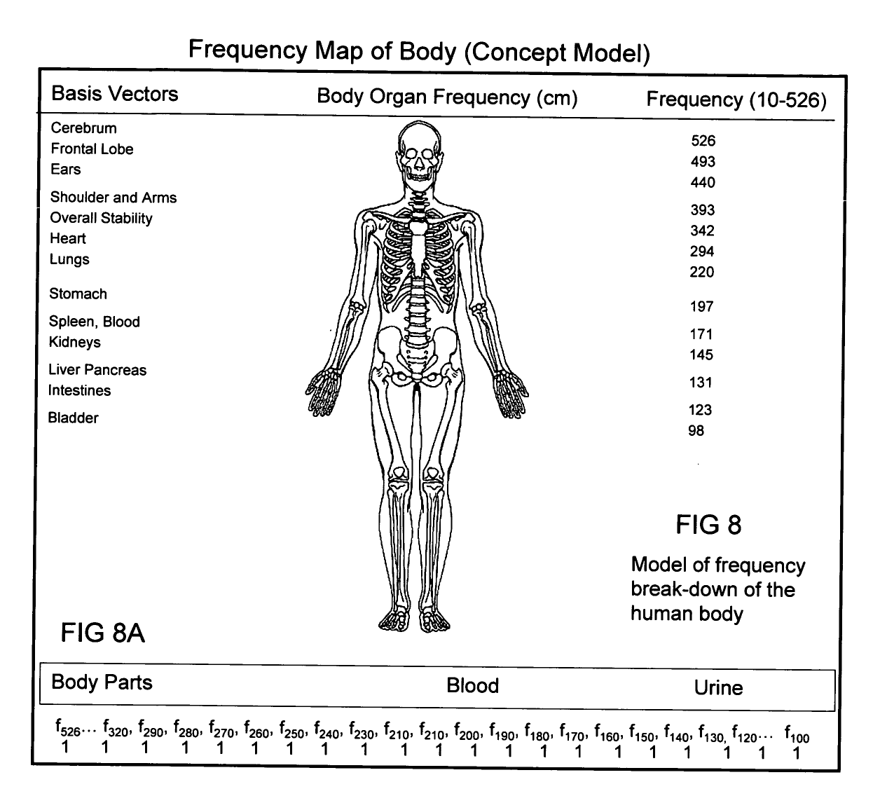

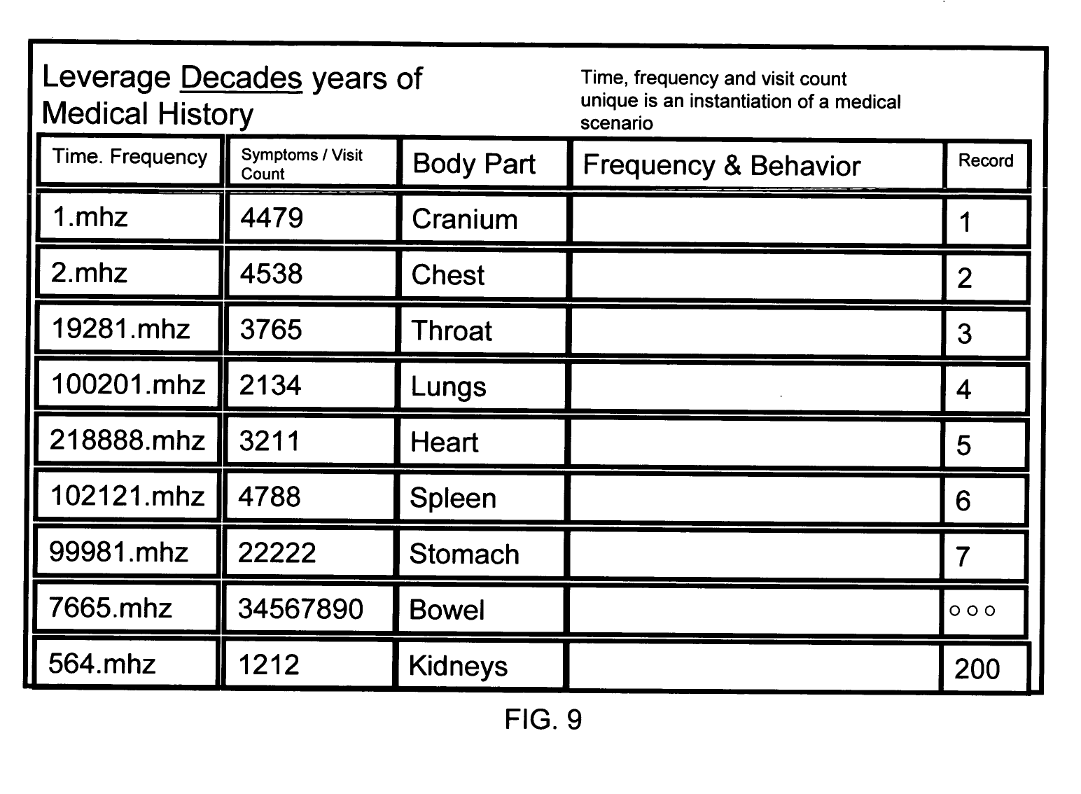

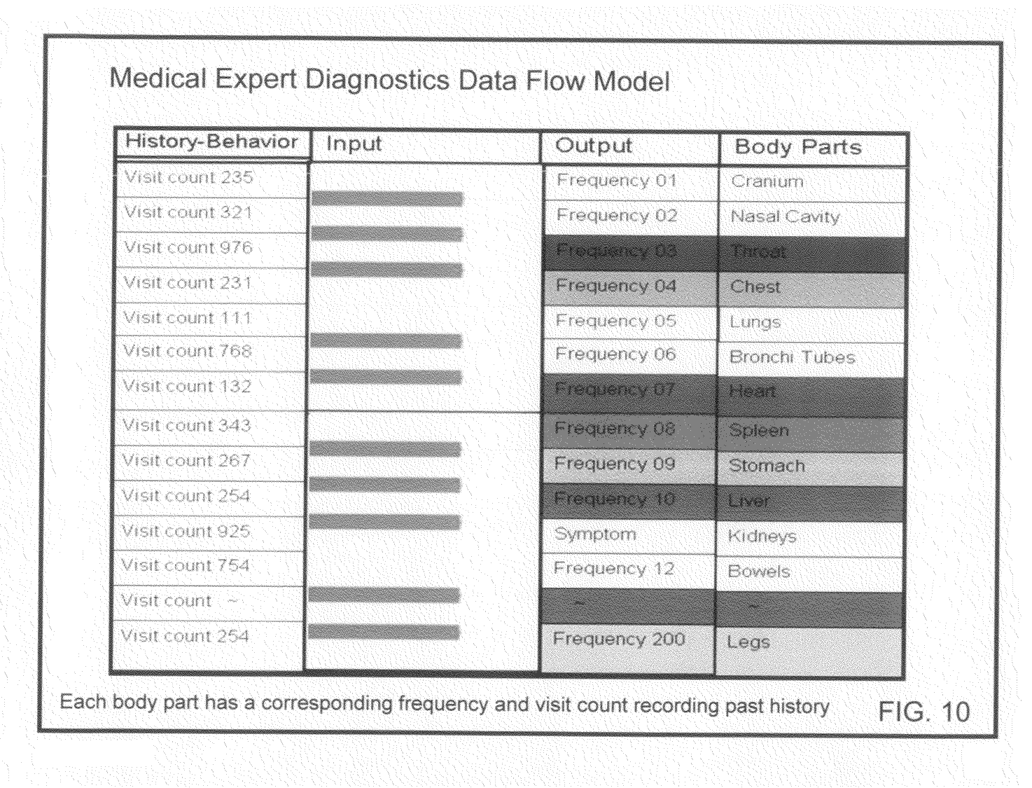

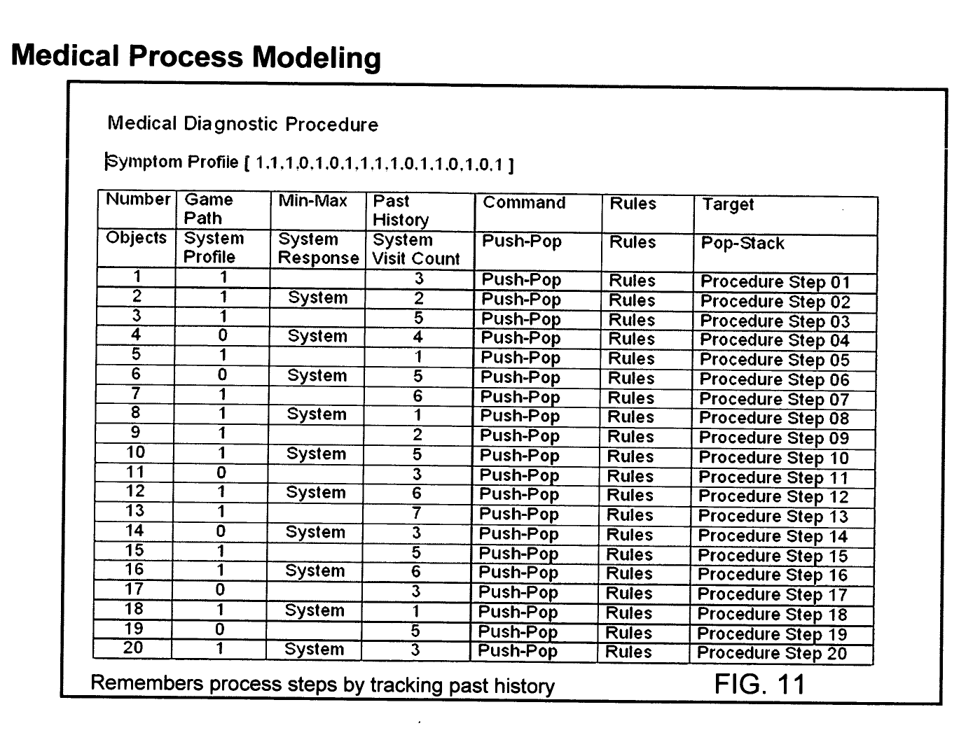

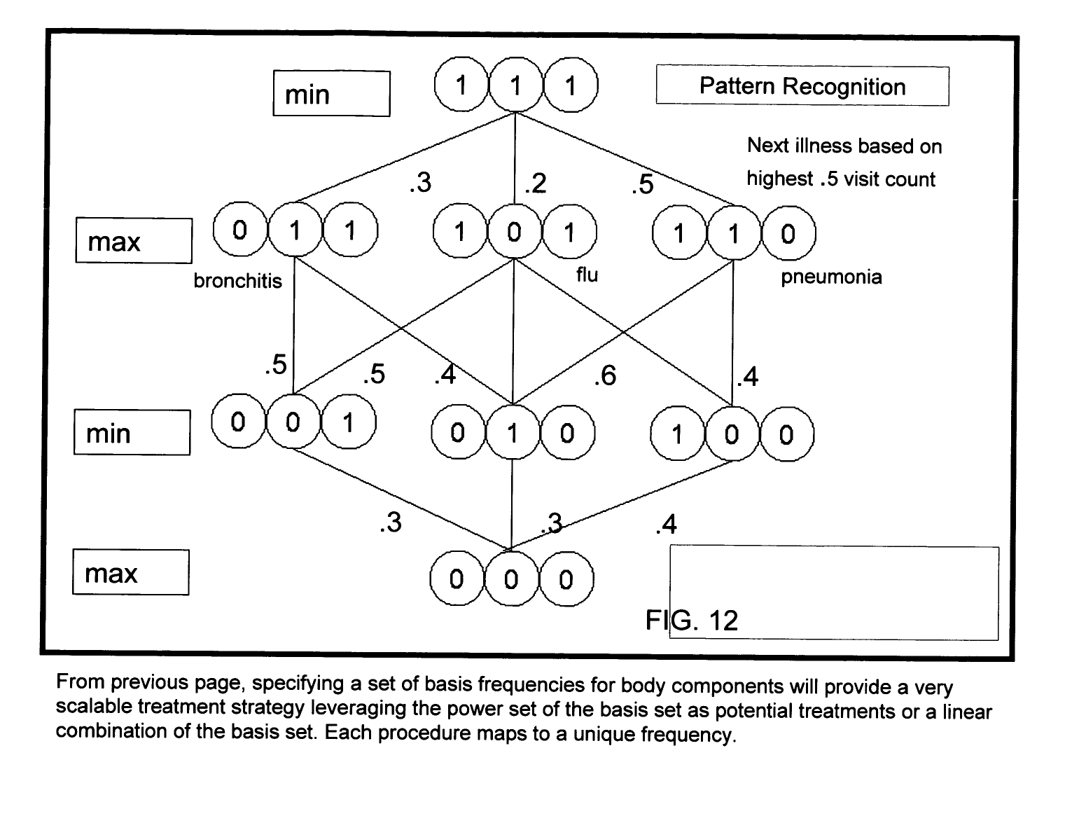

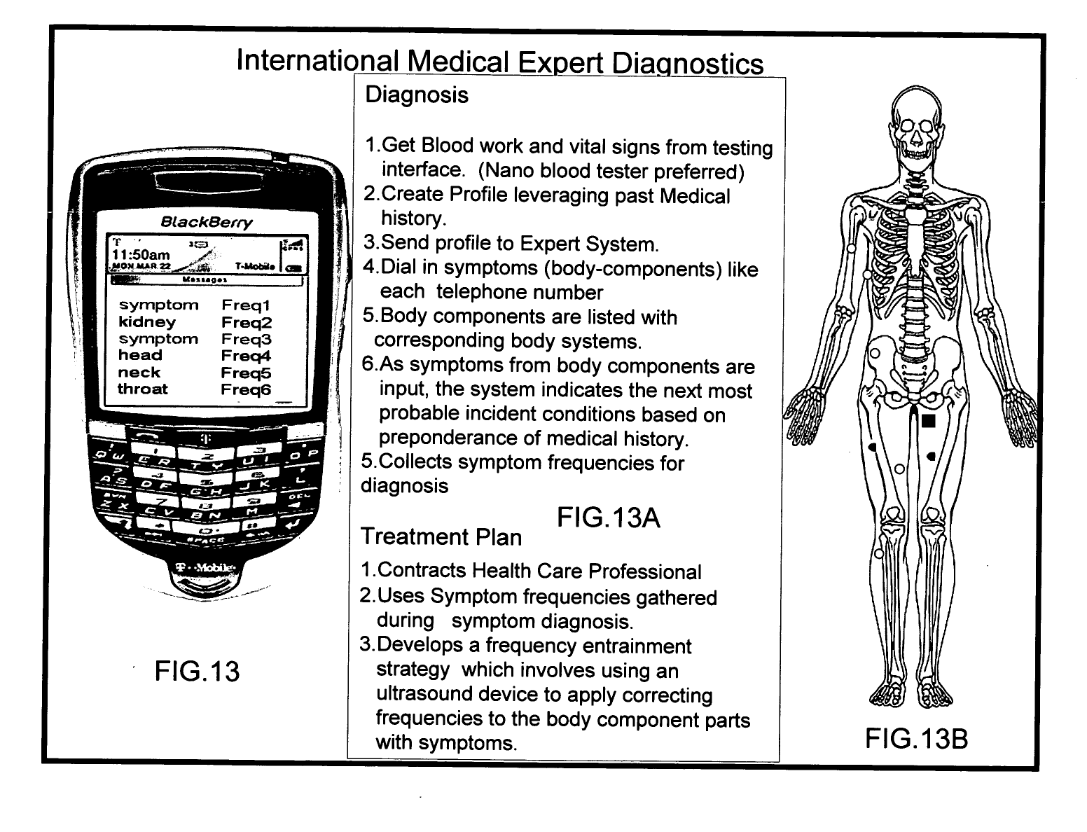

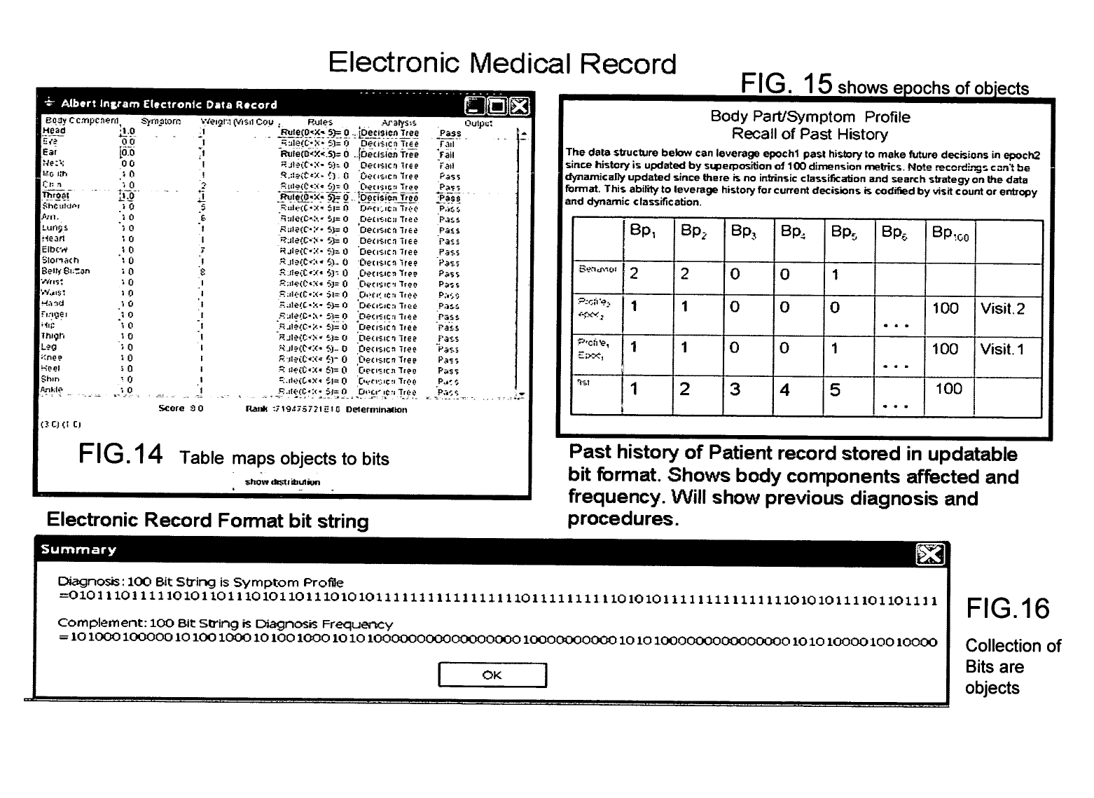

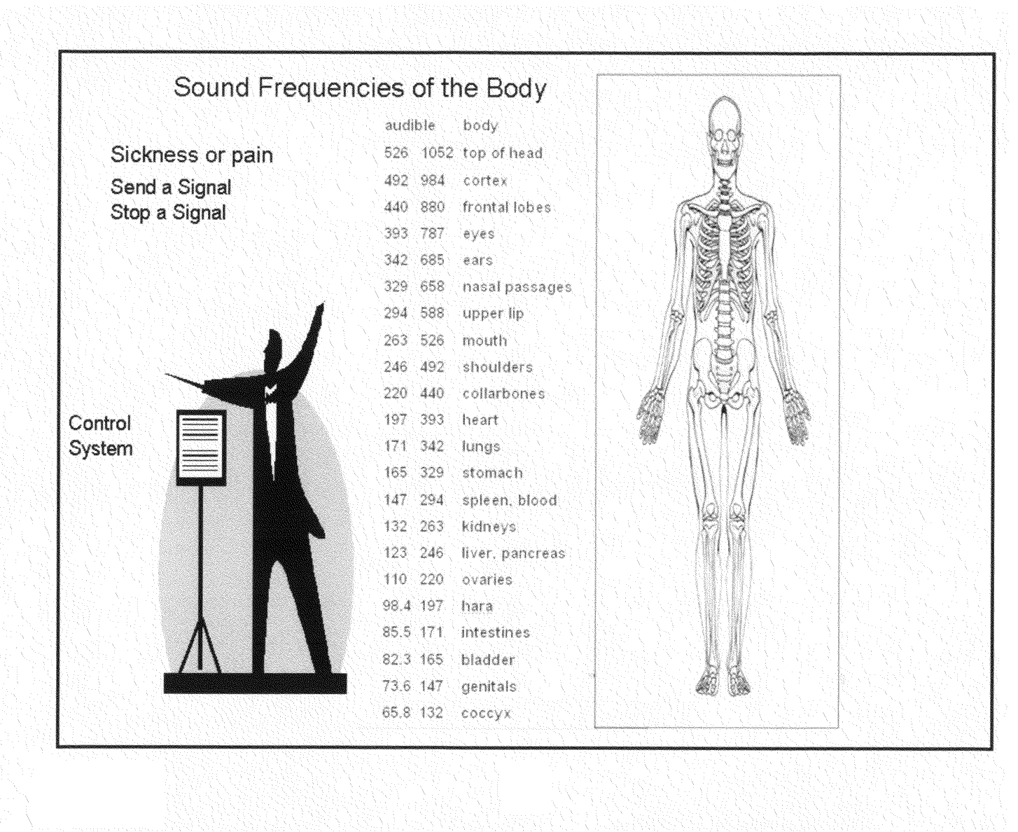

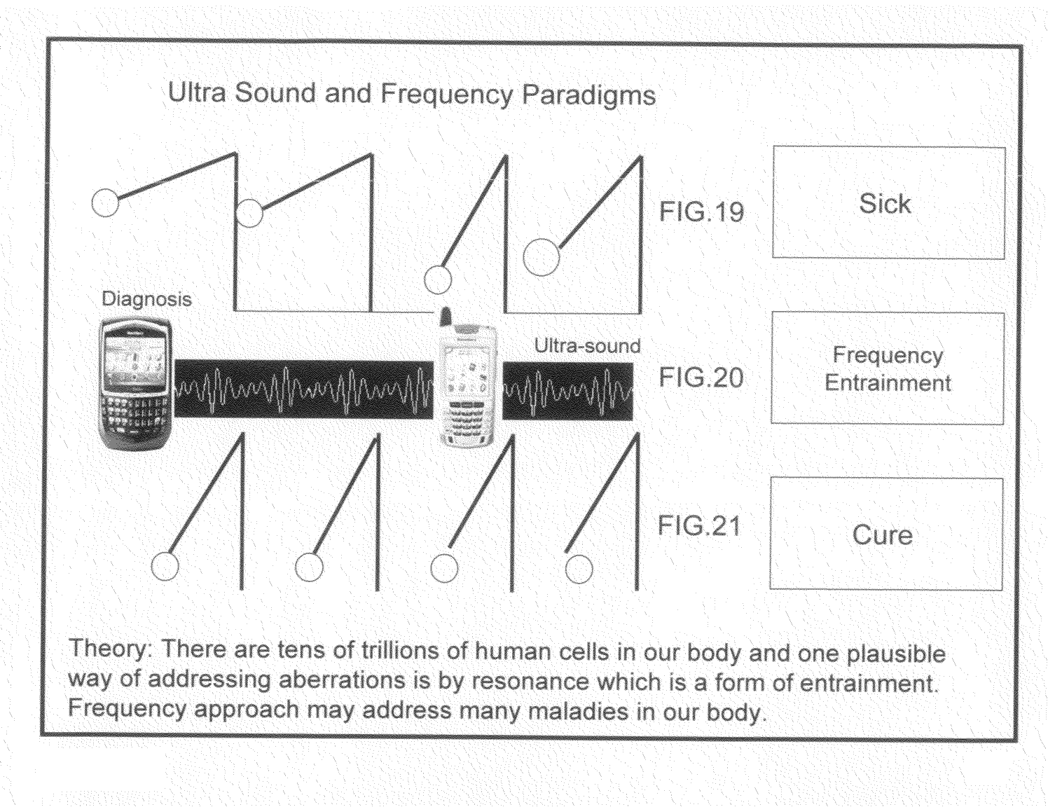

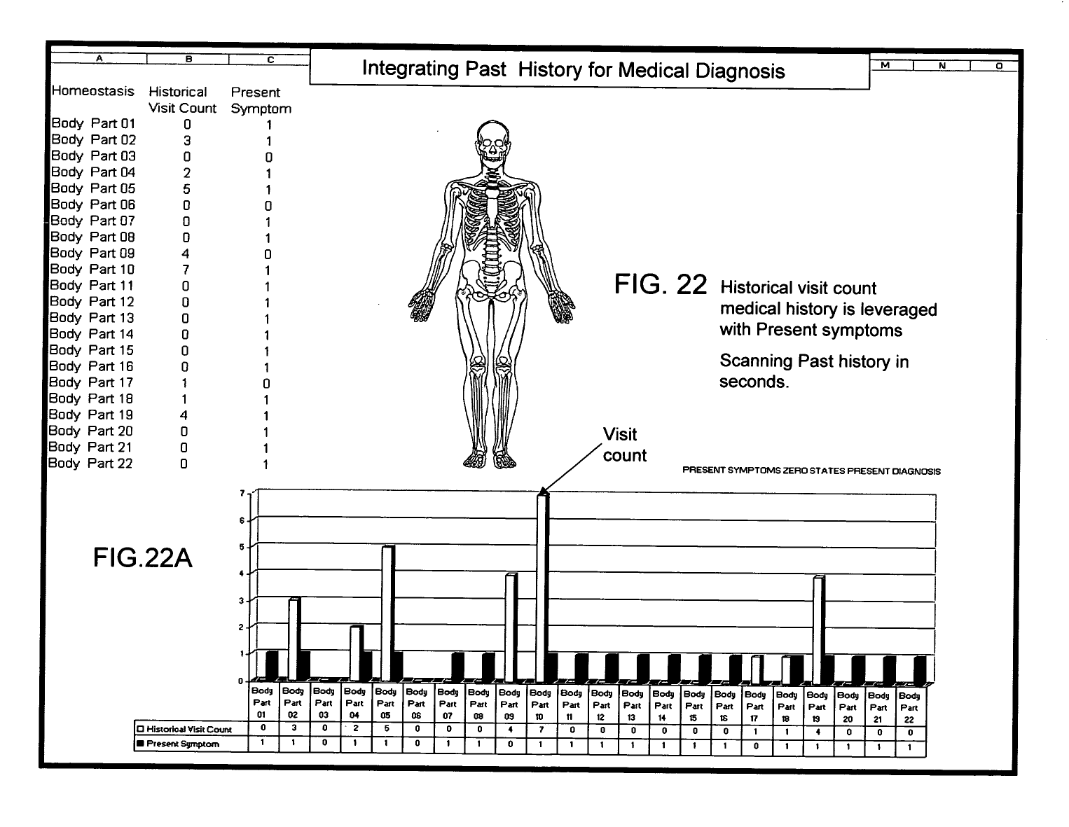

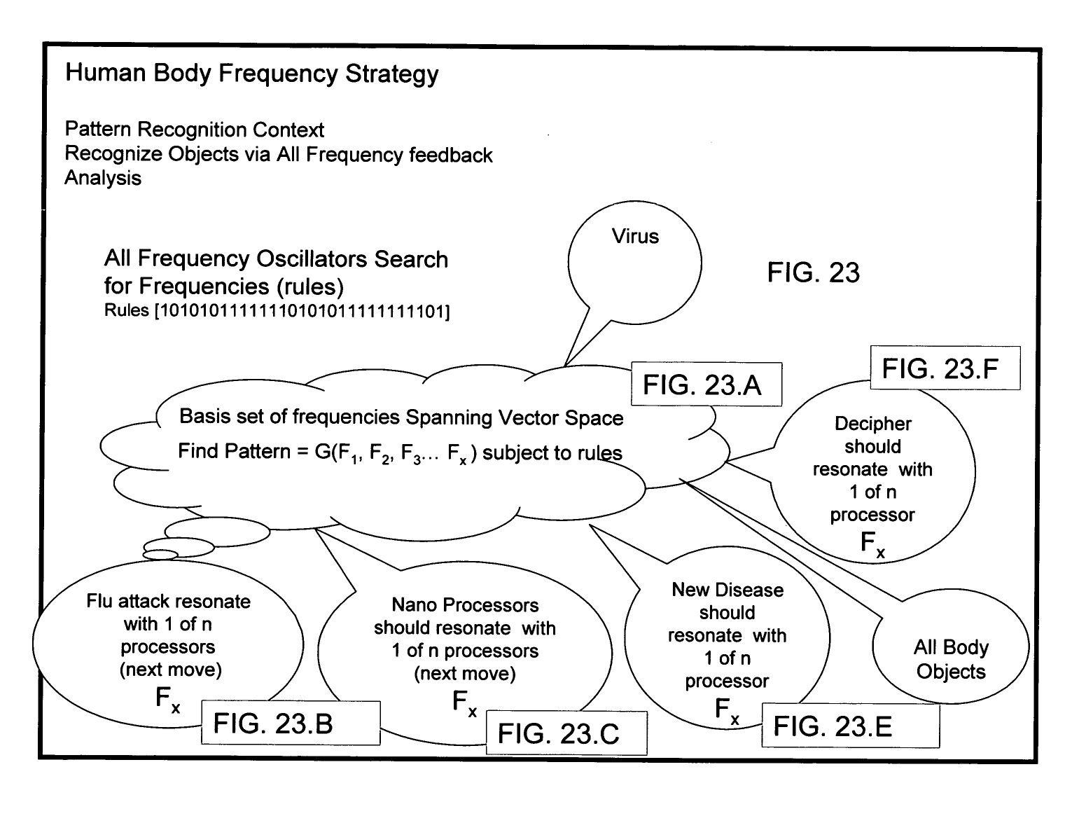

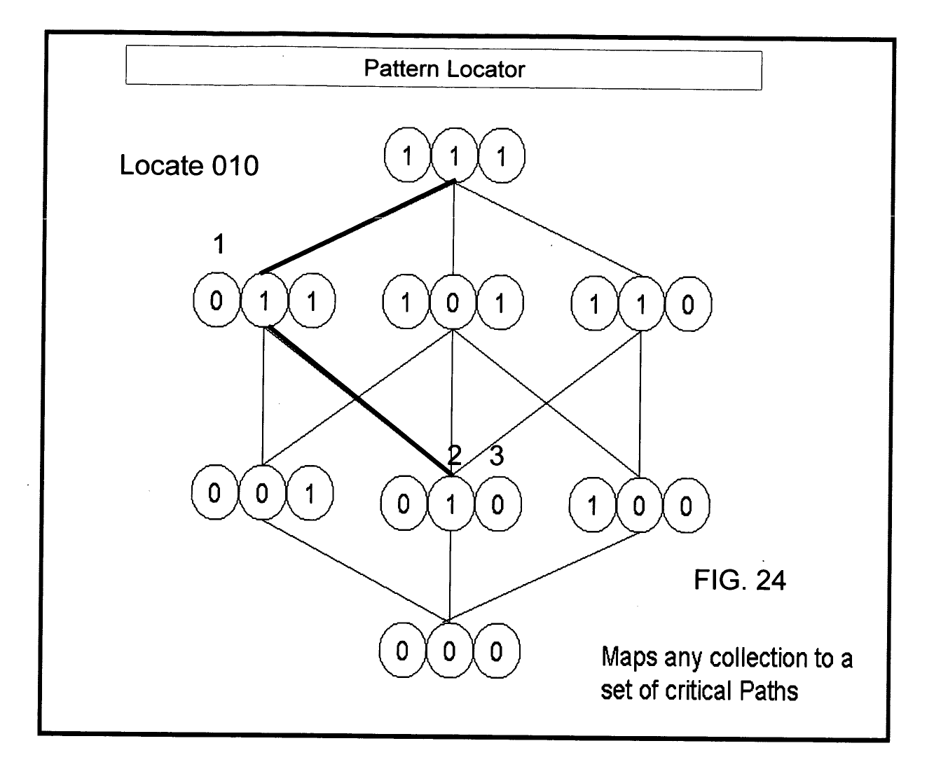

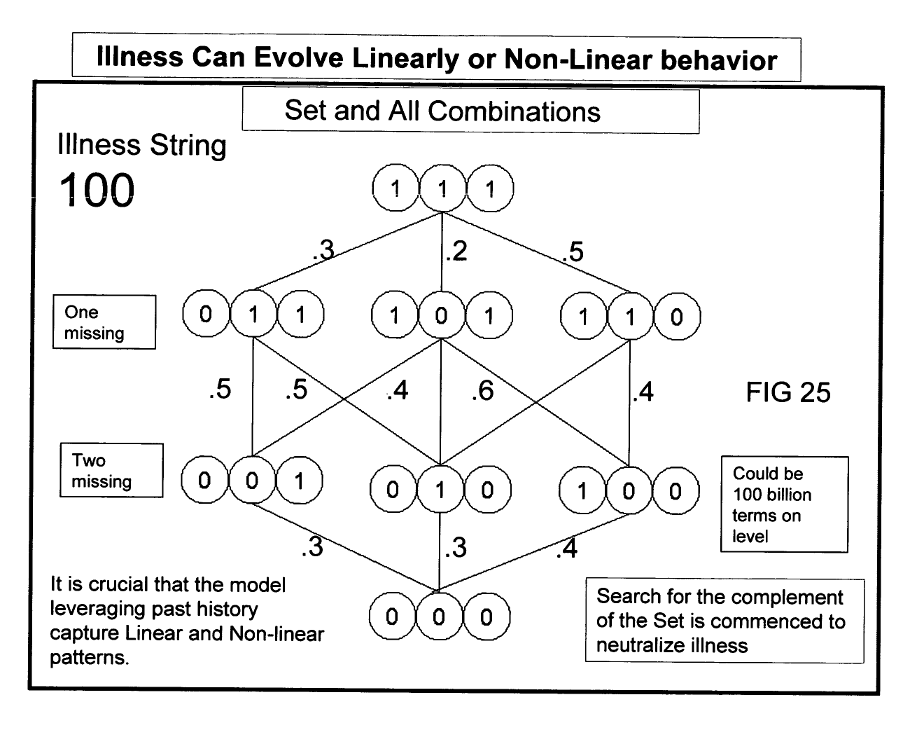

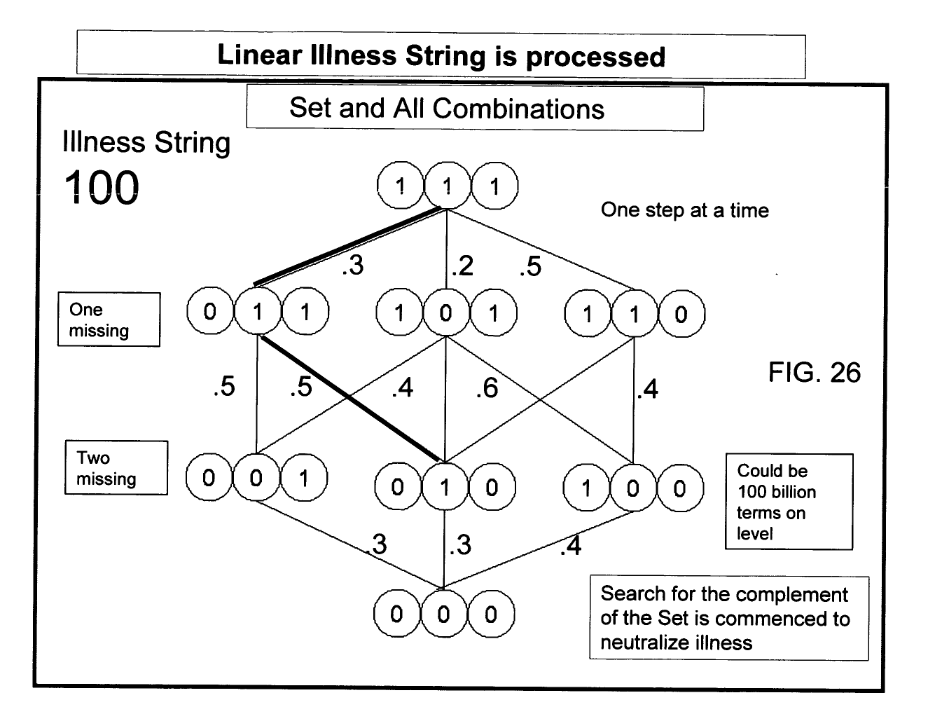

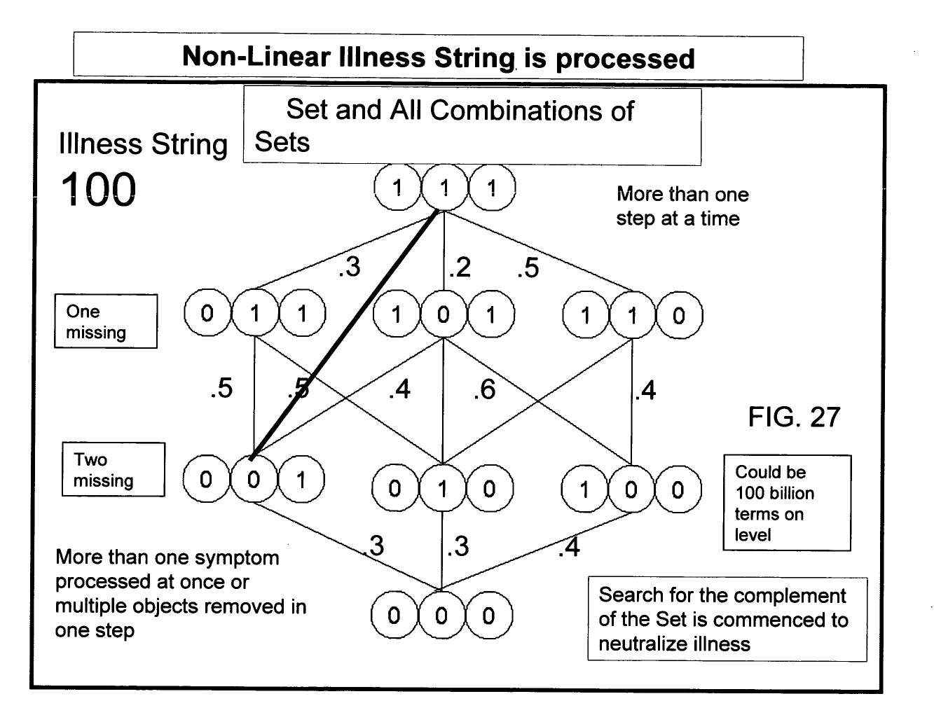

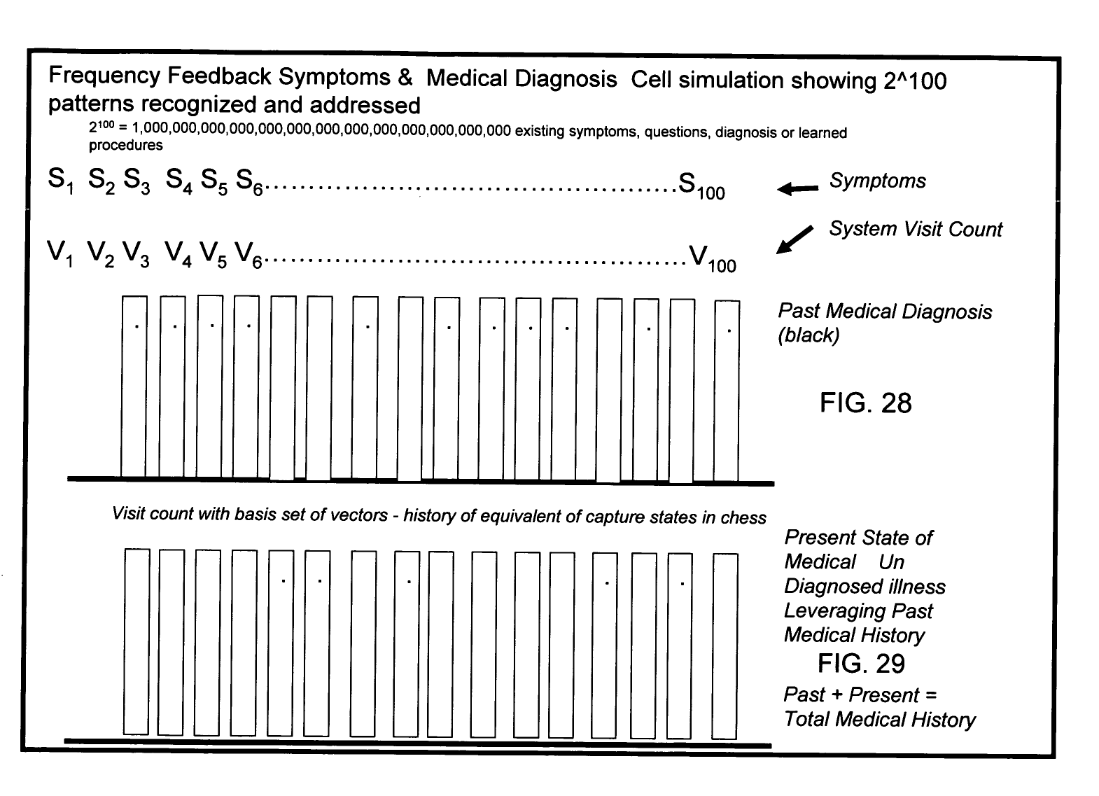

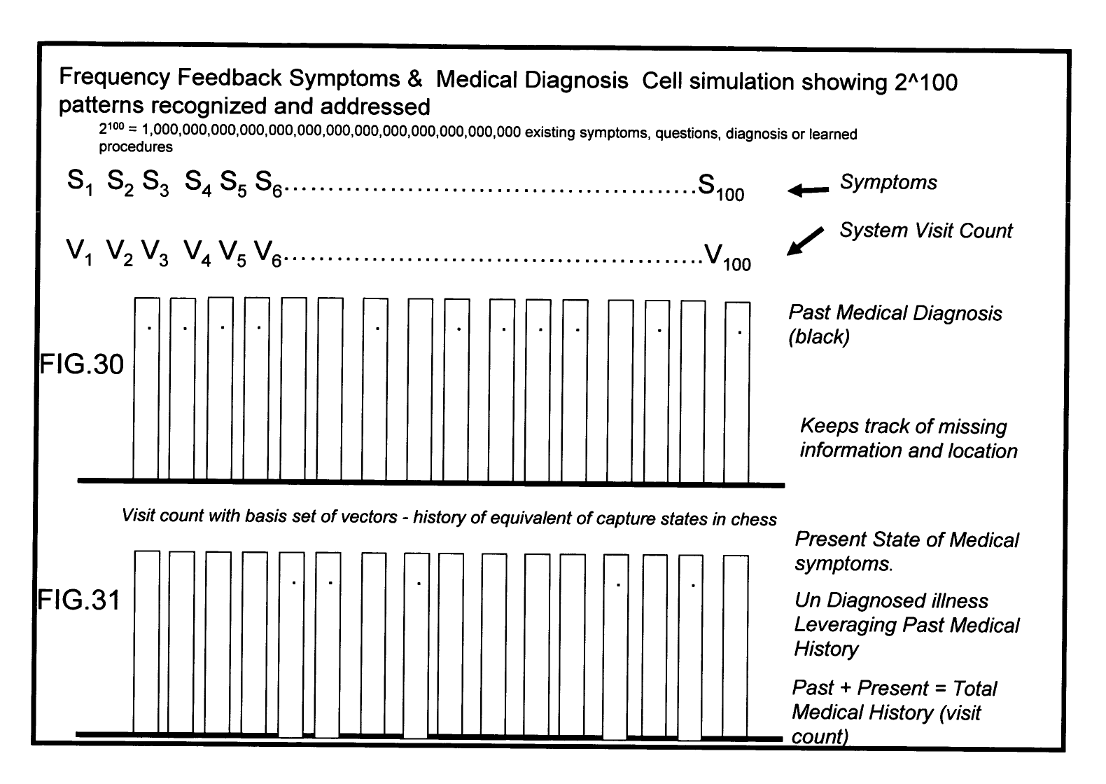

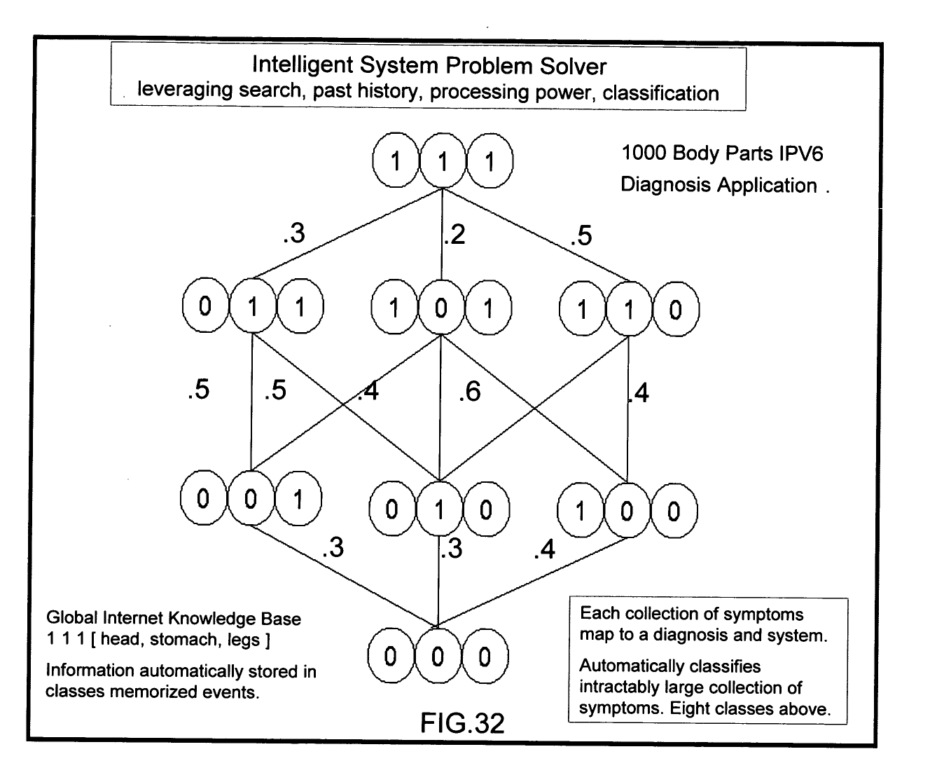

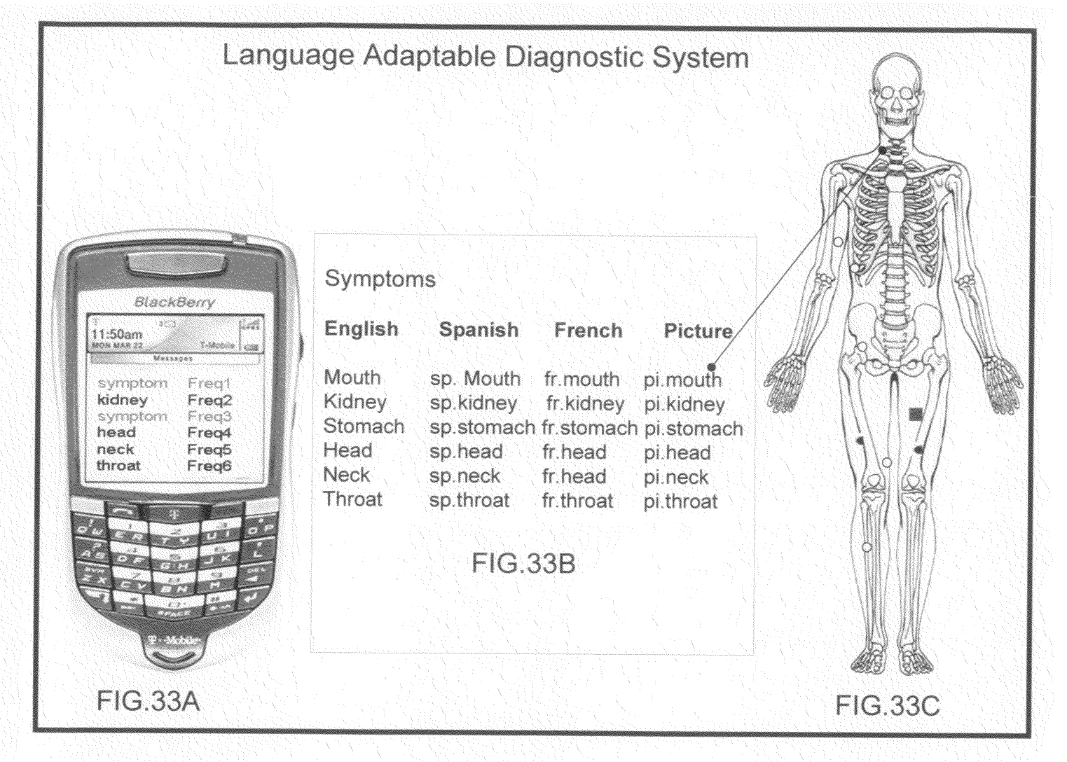

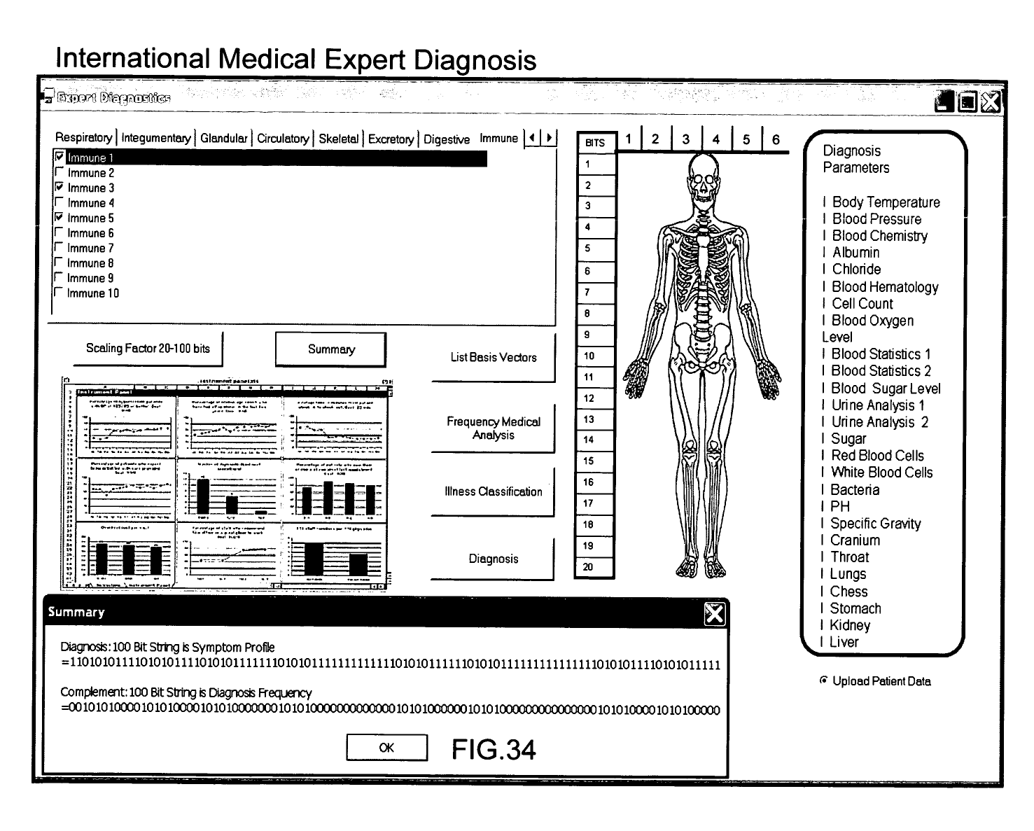

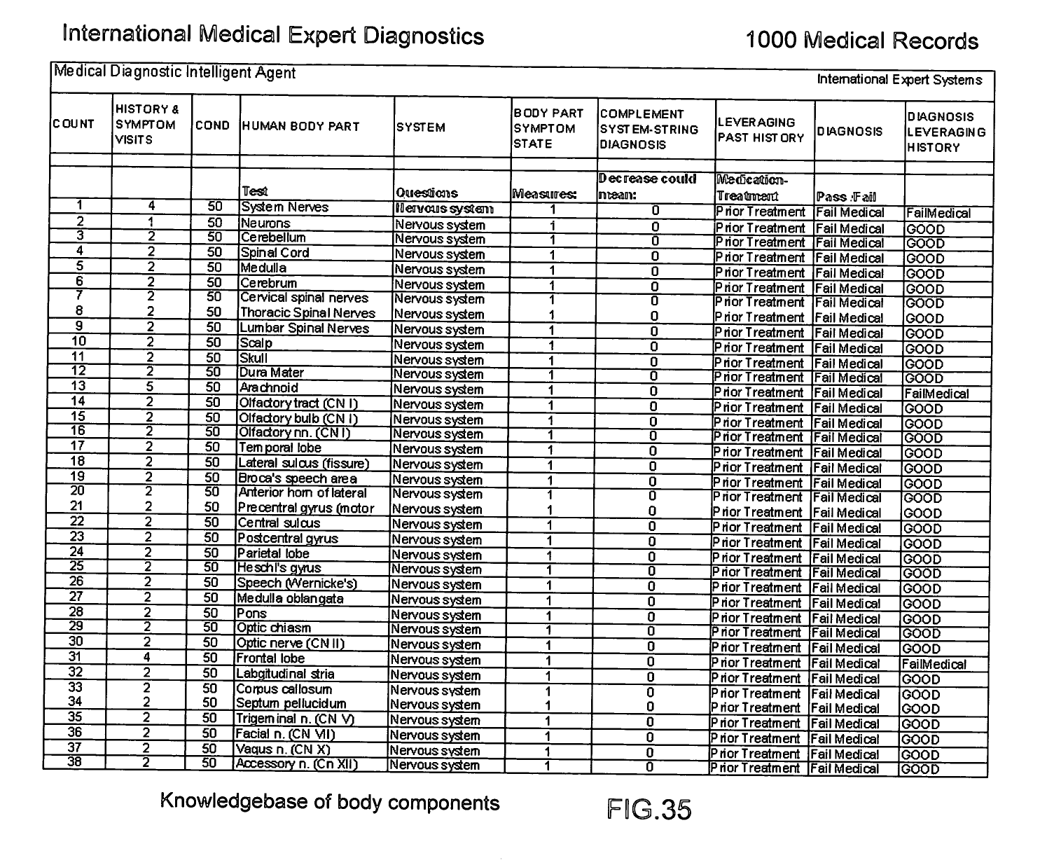

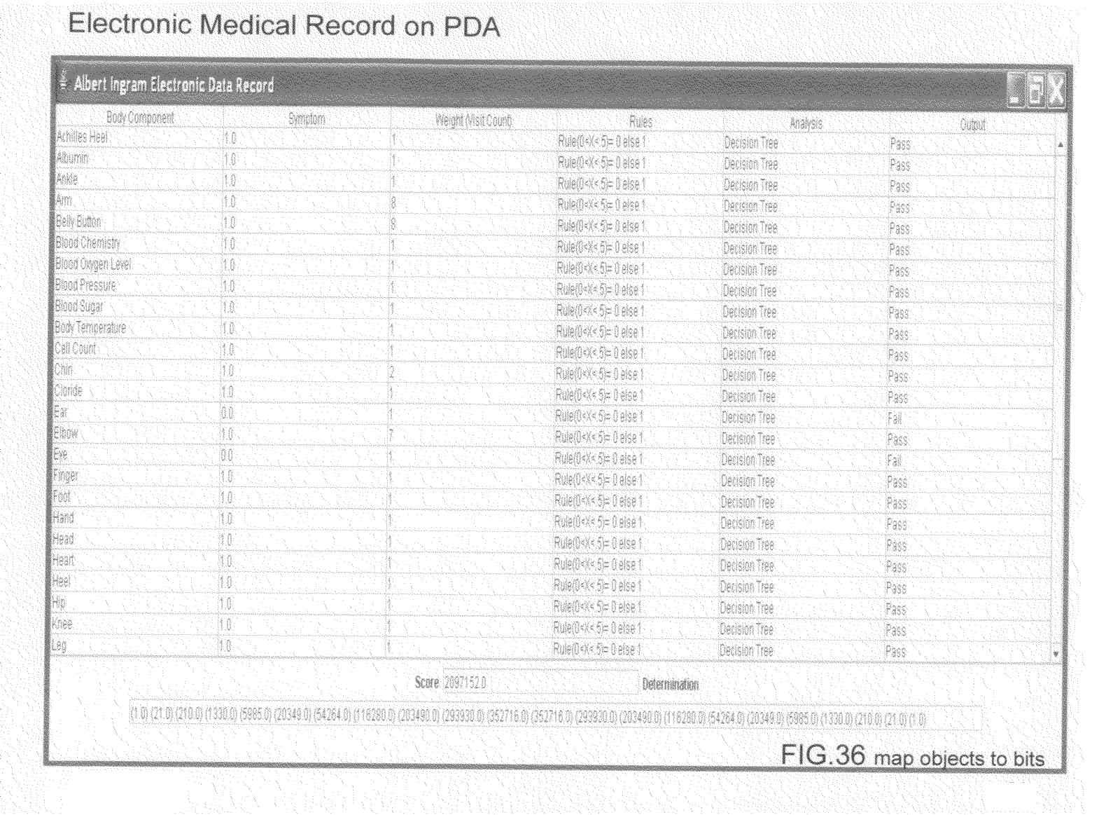

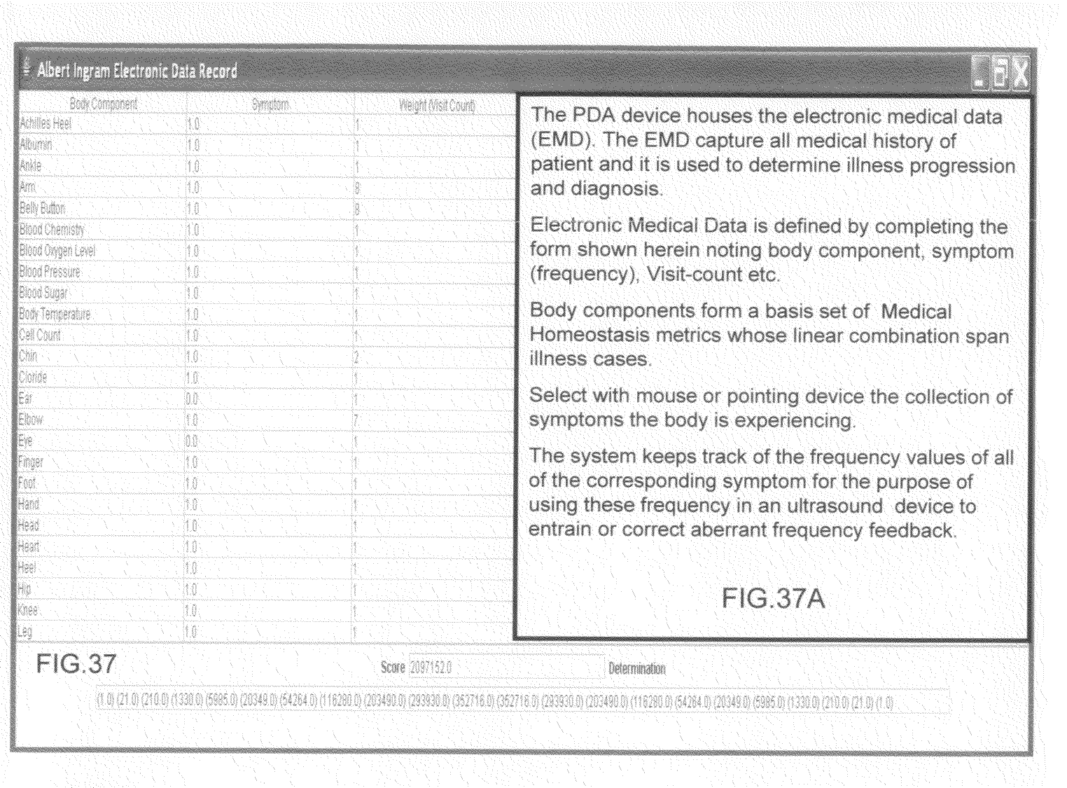

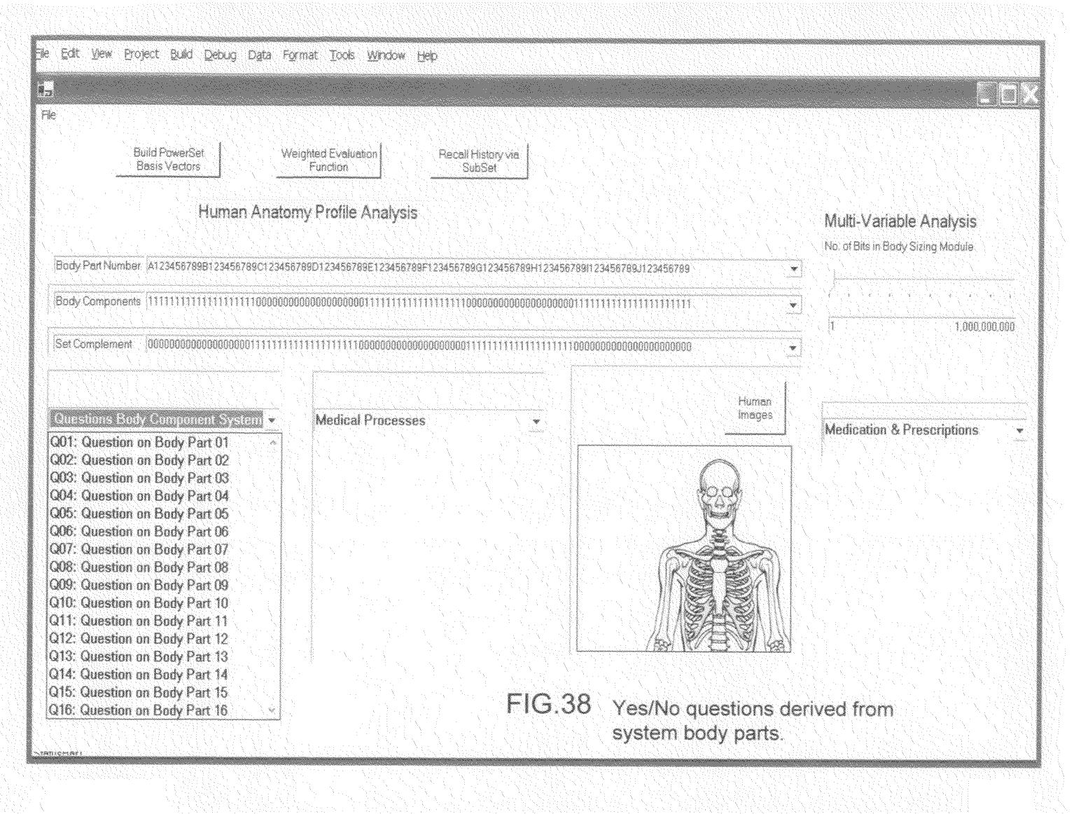

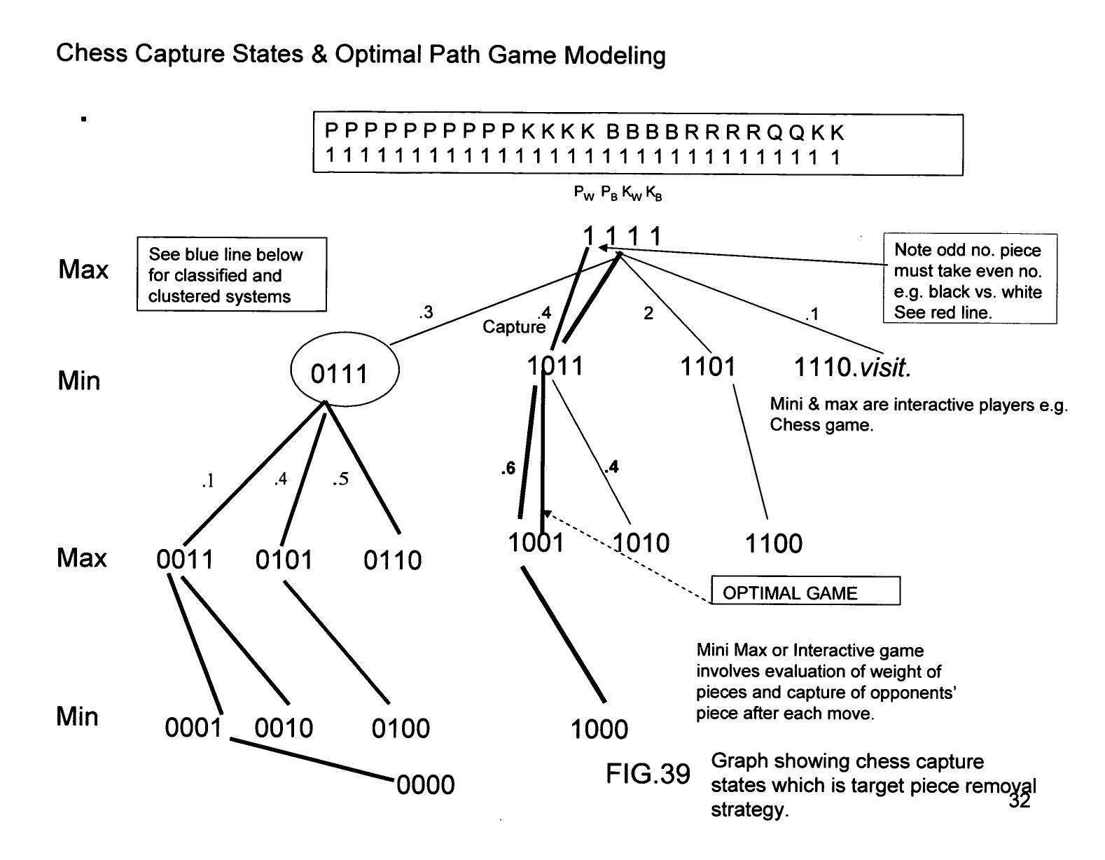

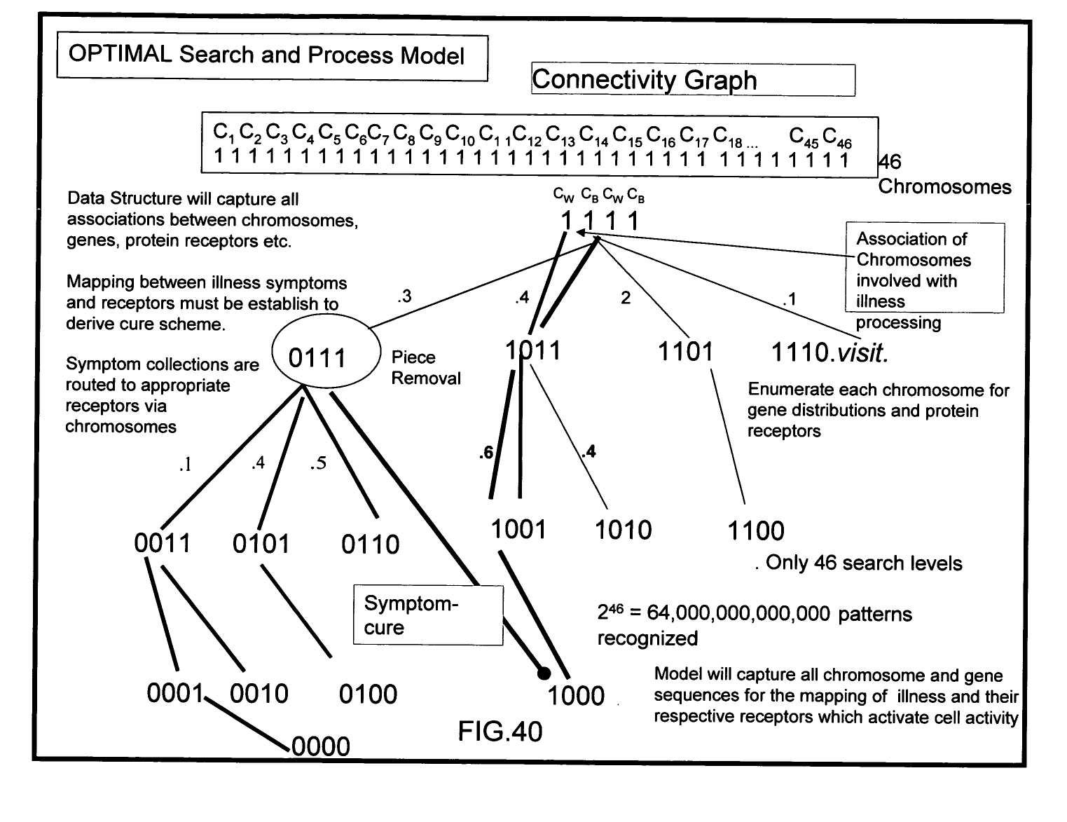

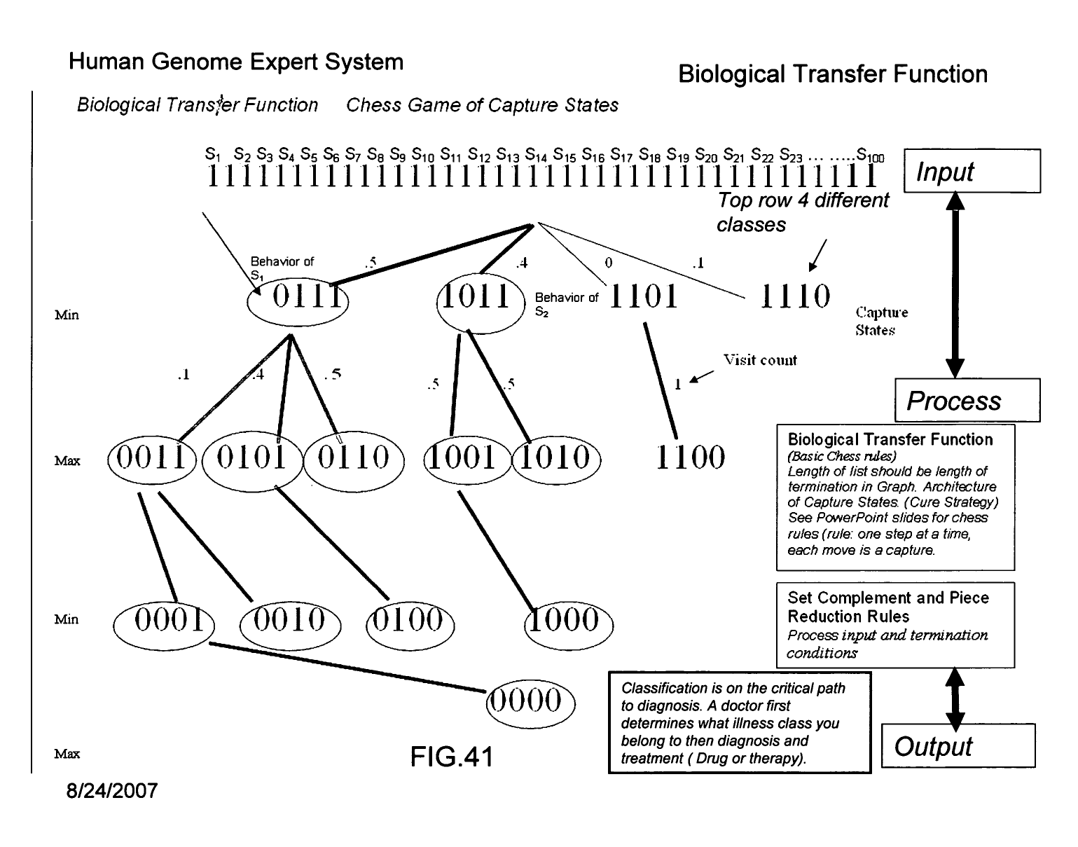

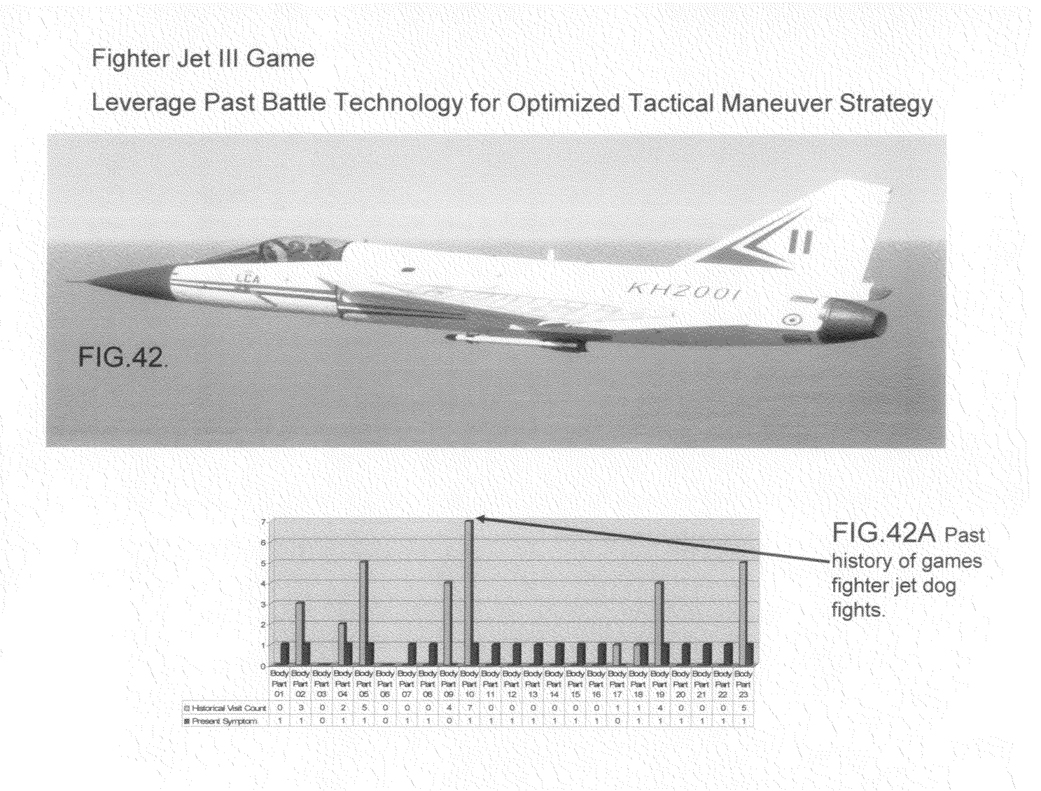

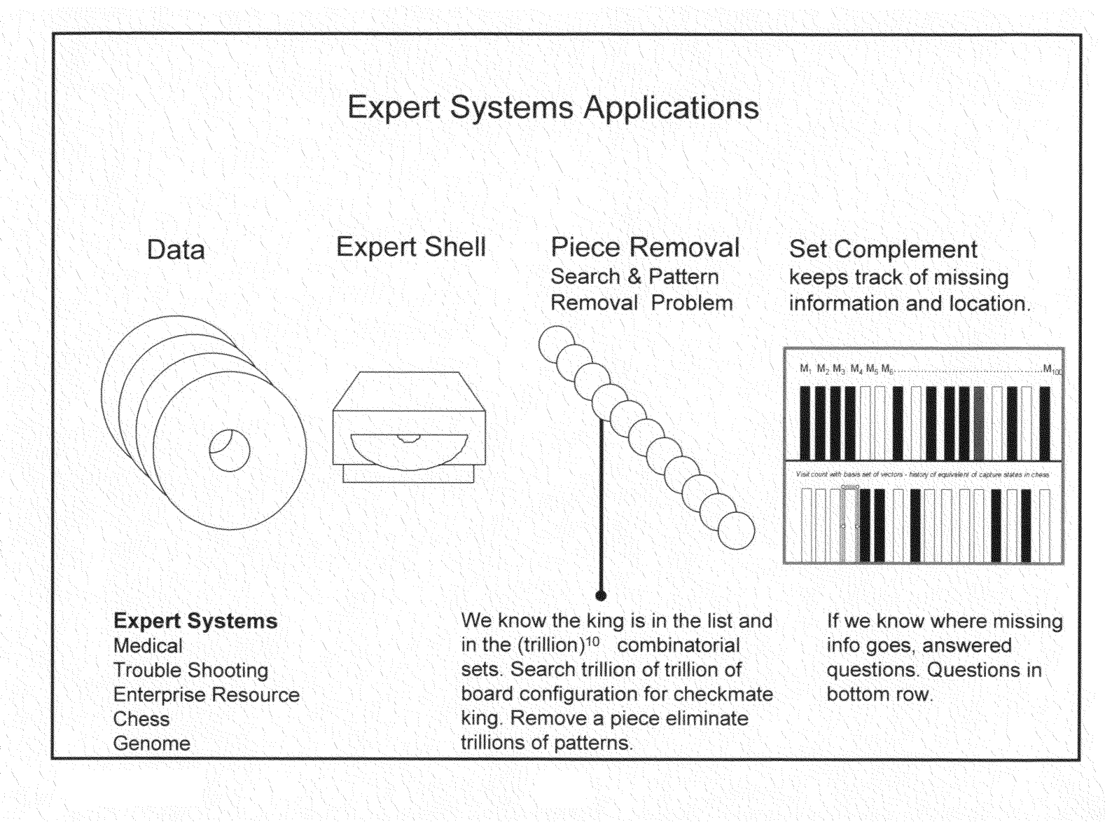

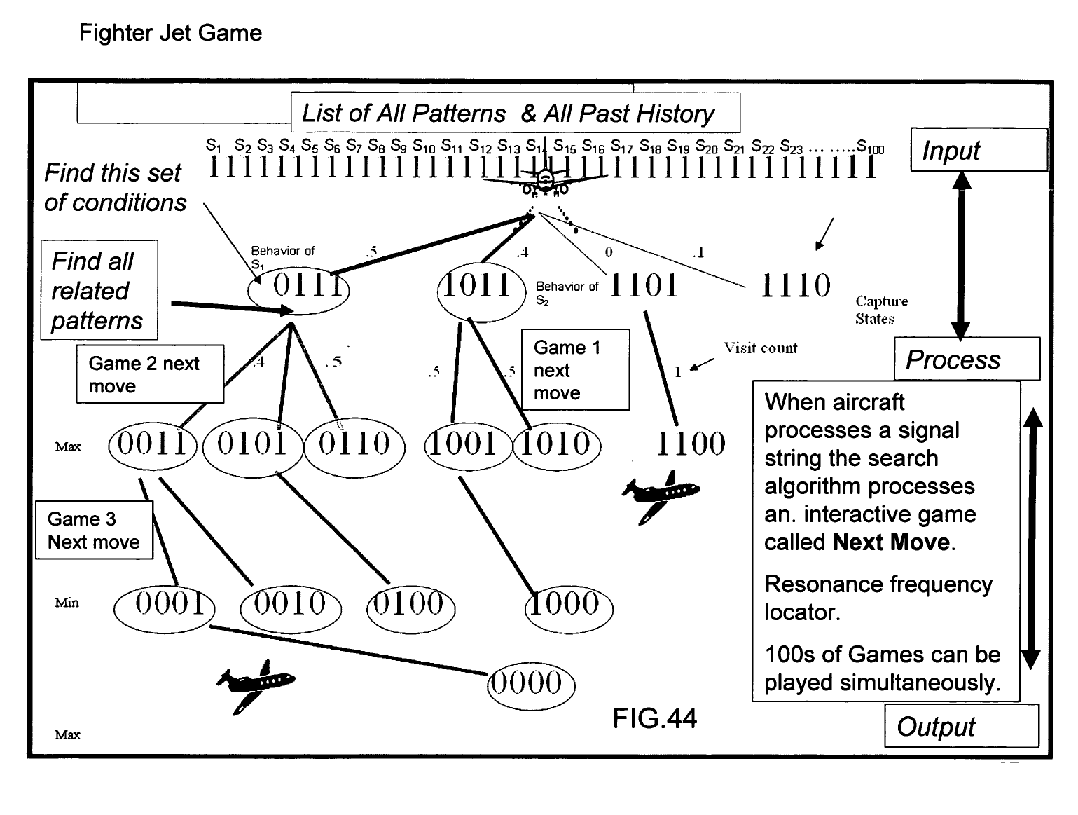

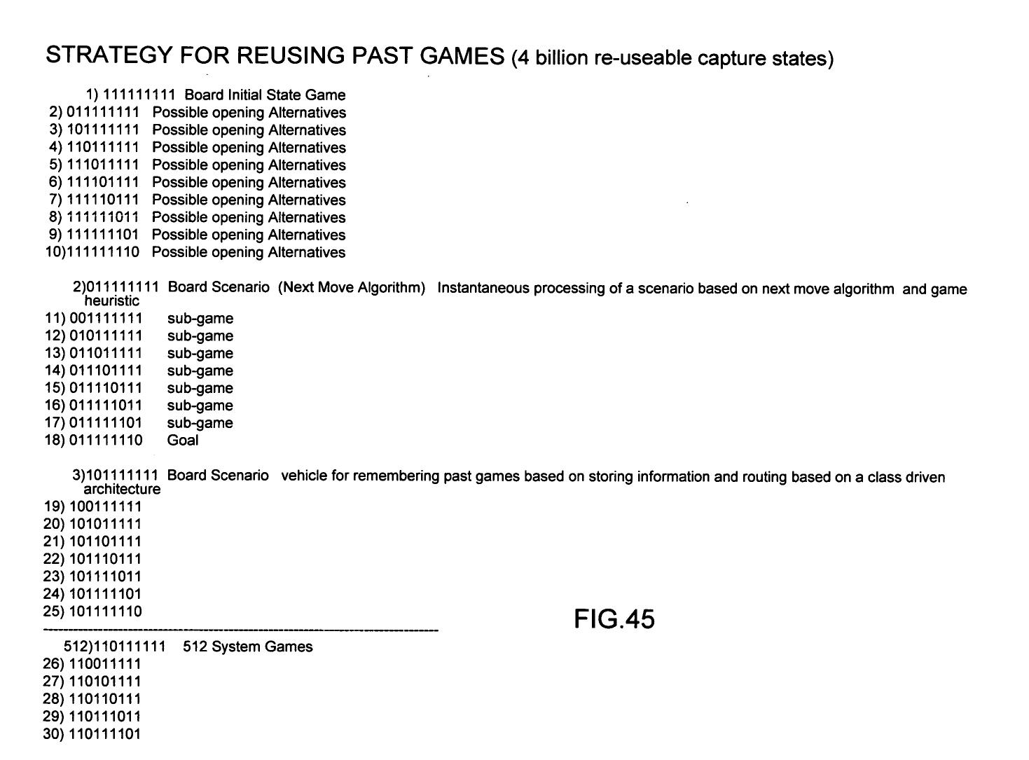

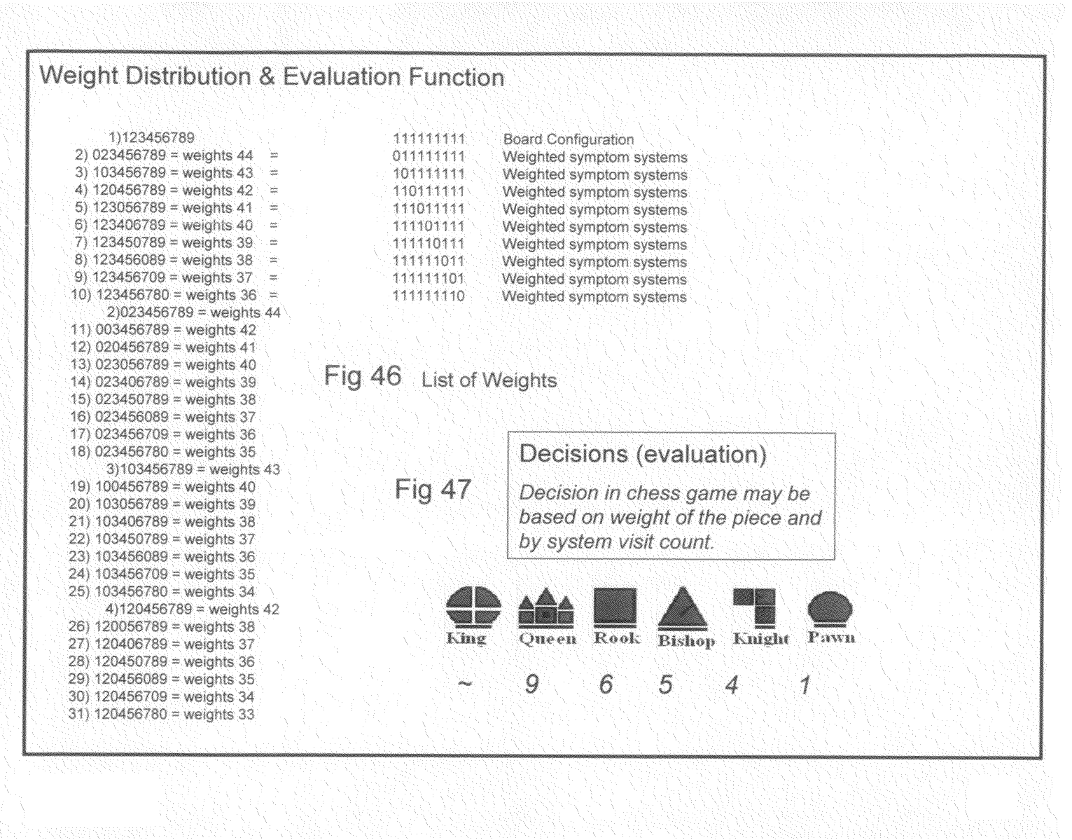

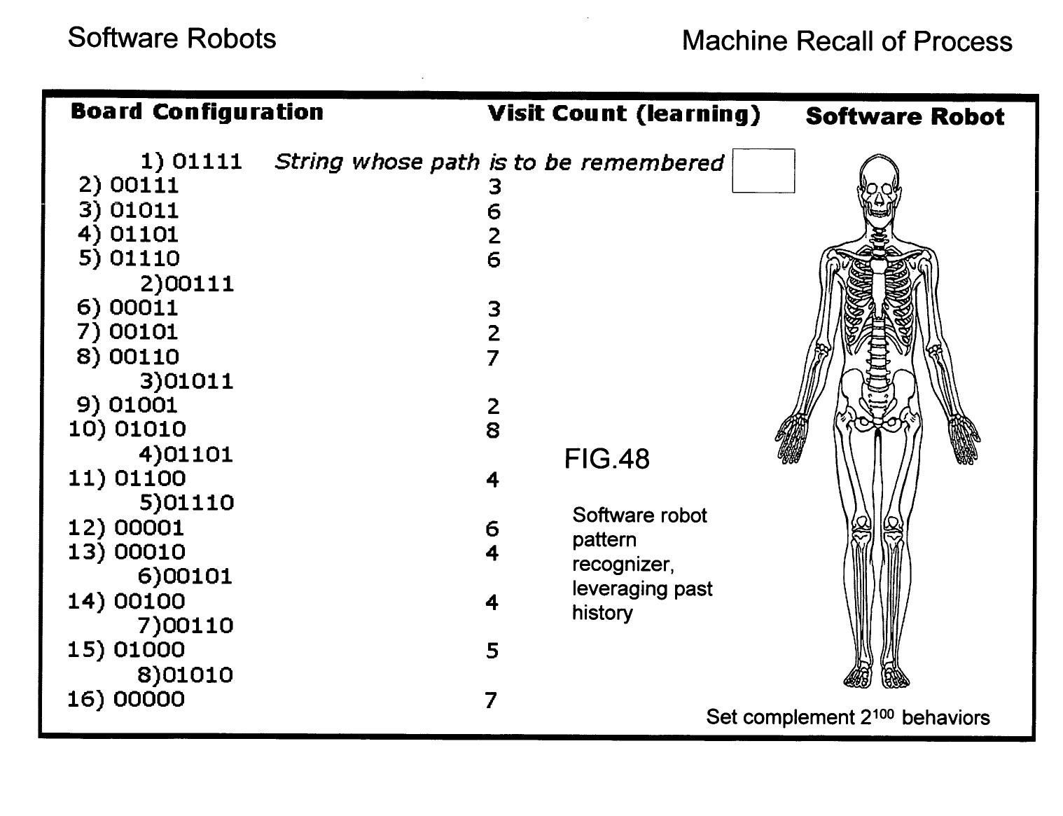

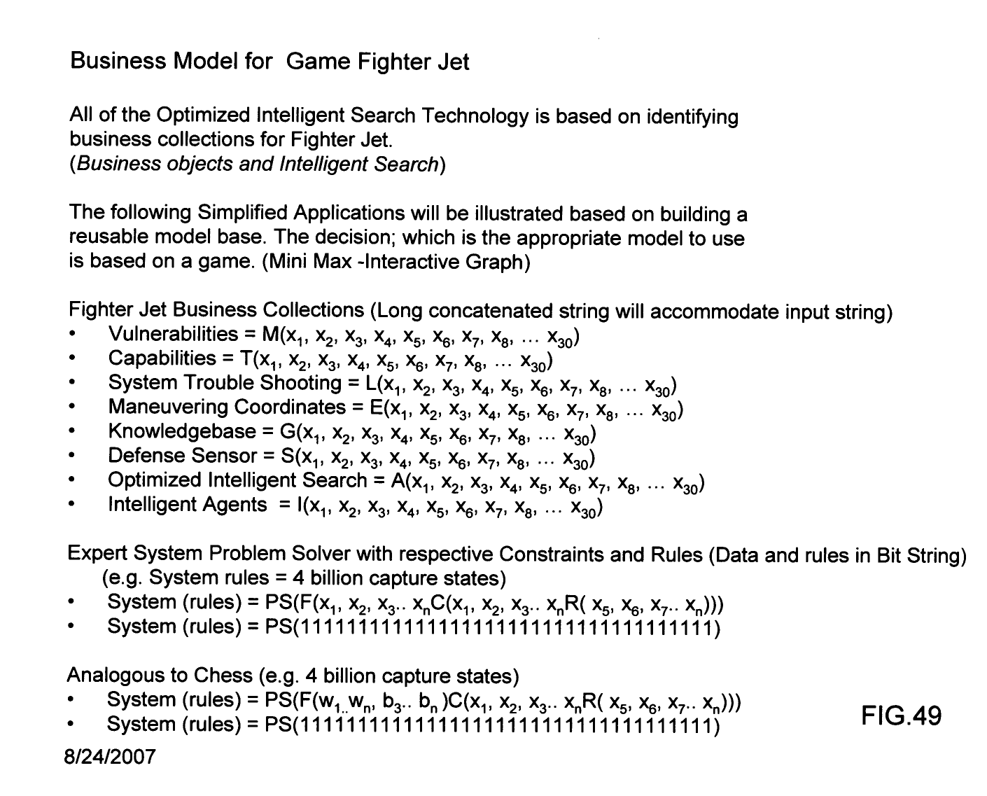

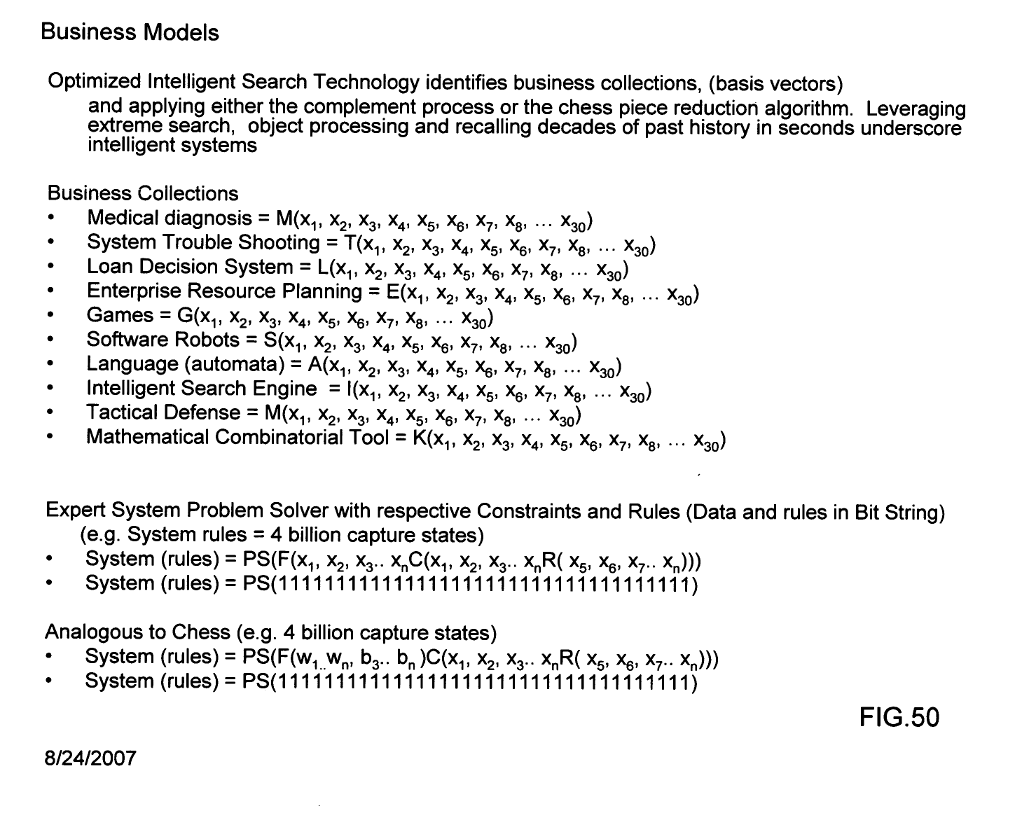

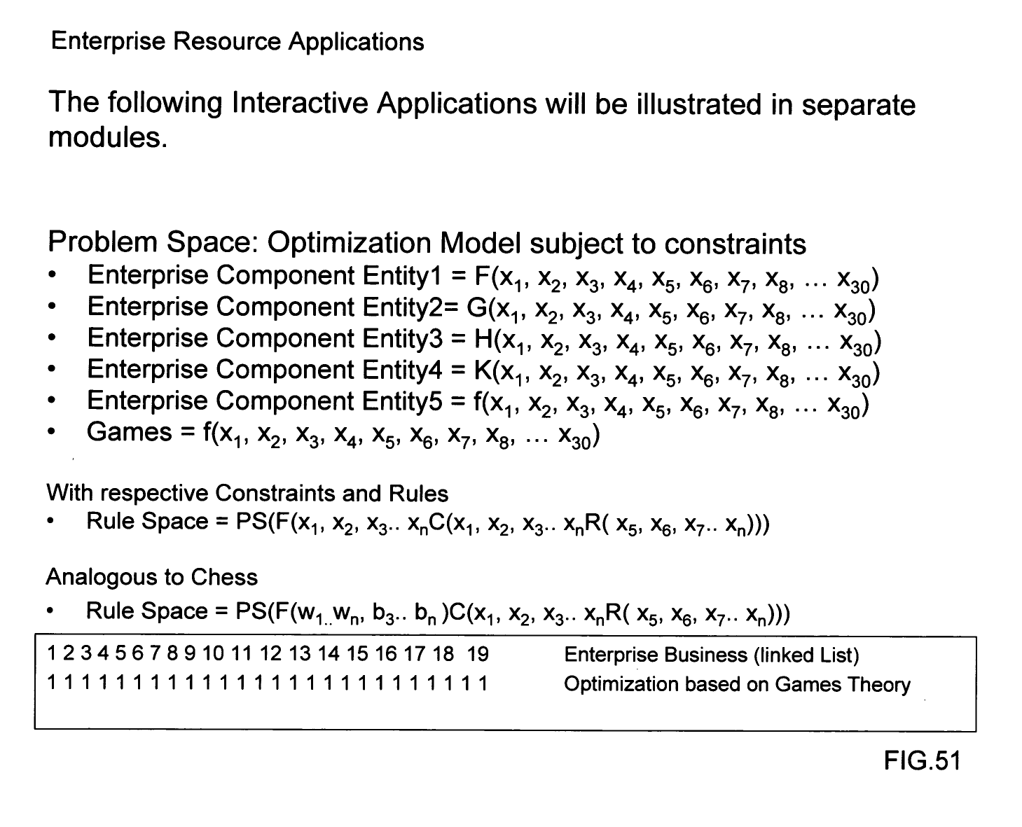

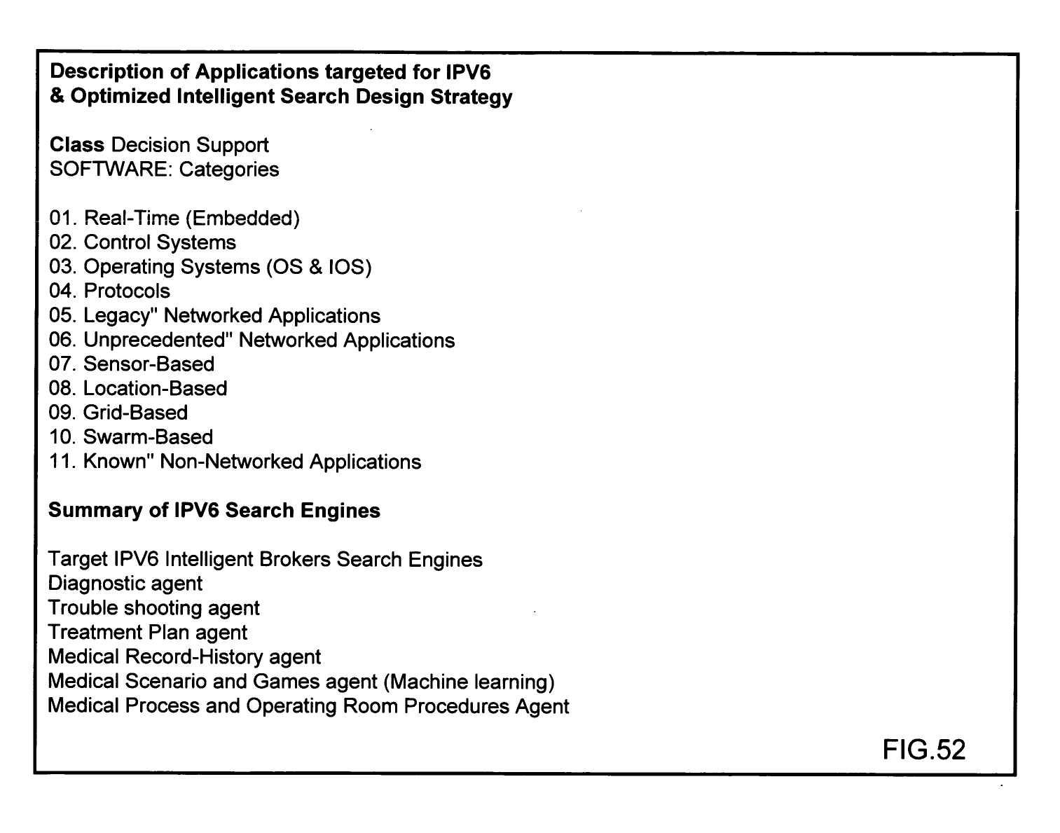

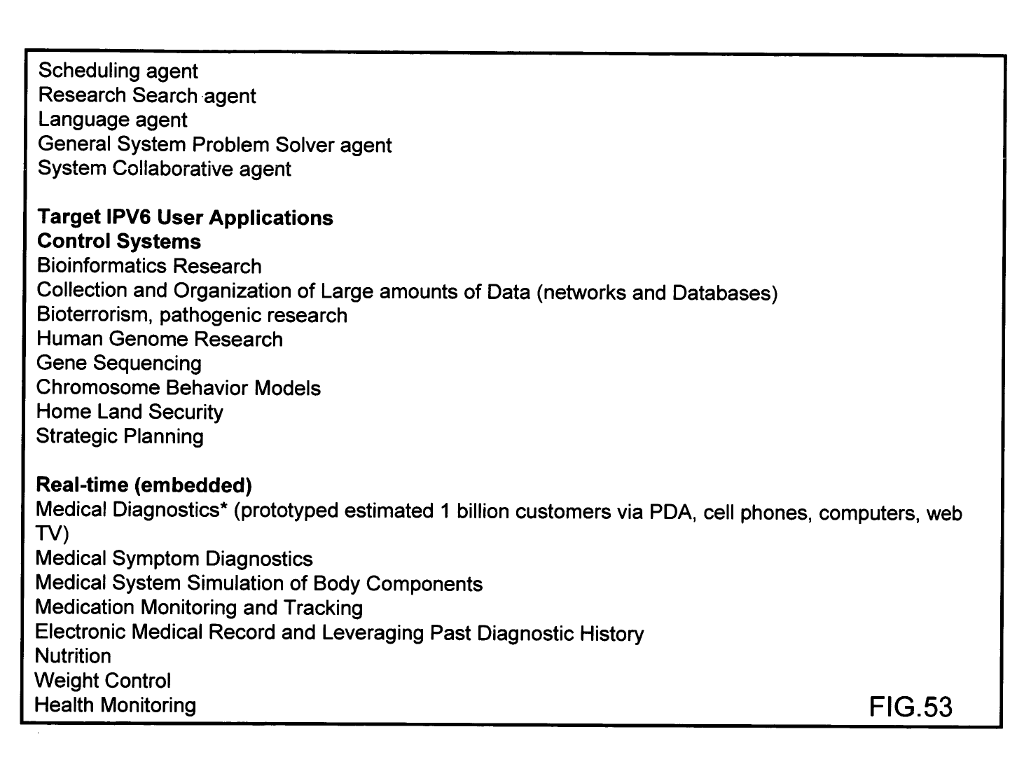

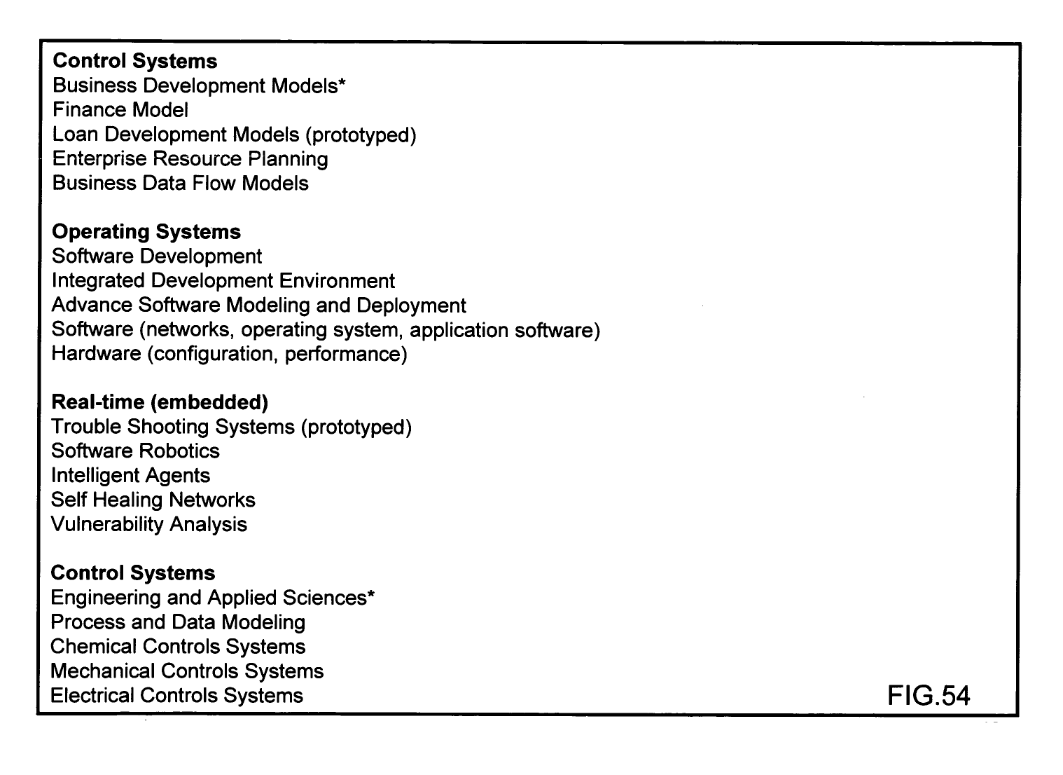

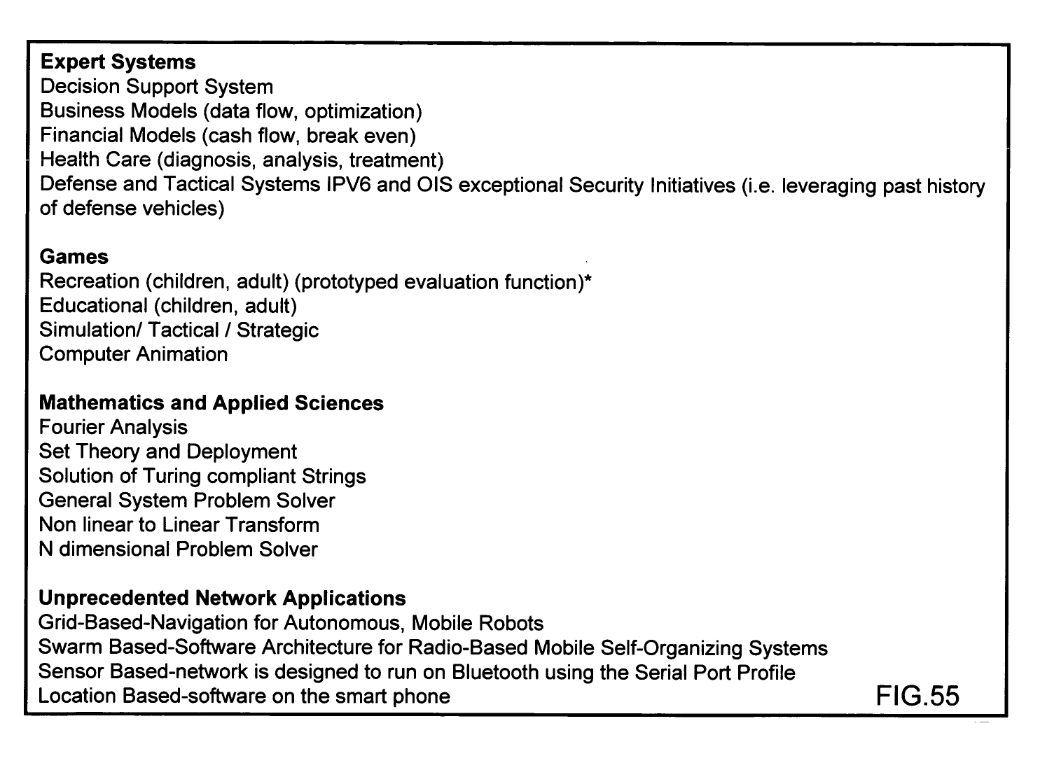

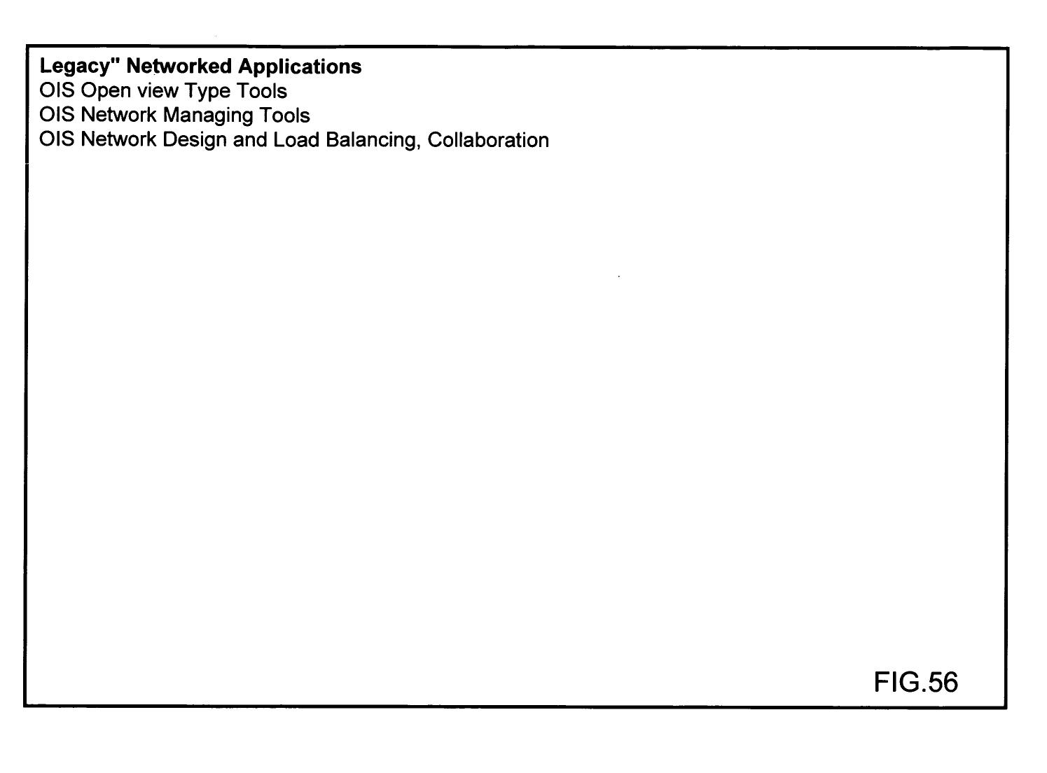

FIG. 1. Data The user of the expert system must define a collection of basis objects which span the problem space. It is assumed that a linear combination of any set of these basis objects will correspond with any instantiation in the domain. Typical collection of basis vectors for Expert Systems Medical, Trouble Shooting, Enterprise Resource, Chess, Genome are derived by experts or system professionals.

FIG. 2. Expert Shell reusable container in the expert system (minus the basis vectors) which facilitates the search for the correct set of states which identifies a set of aberrant conditions or in the case of symptoms a medical diagnosis. An expert system answers the questions the customer asks.

FIG. 3. Piece Removal facilitates Search Problem by removing objects from game tree (e.g. capture in chess game or pattern reduction strategy. It appears that piece reduction in a games venue is one of the unparalleled search strategy mainstreamed.

FIG. 4. Set Complement determines all missing data from the target set or basis set of objects. It is a set operation which keeps track of missing information and object location. In performing diagnosis or system analysis on a system we assume the initial state of homeostasis defines a well working collection of components. As system inconsistencies and flaws arise, the diagnosis process keeps track of all the original start states of the system, collects all the system aberrations or symptoms and identifies the set of objects which needs to be restored to make the system complete. The forte of the diagnosis process is the ability of keeping track of trillions of trillion of system states leveraging a search process that is fundamentally derived from the chess piece capture process

Piano Analog to Optimized Intelligent Expert Systems

Diagnostic Expert Based on 200 Body Part Reuse

If you know the frequency of 88 piano keys then you know the behavior of all 2̂88 keys. You know all combinatorial instances of keys and their corresponding frequency.

Having knowledge of their frequencies provides the information to rectify the aberrant signals.

If these 88 (200) keys are a basis set of objects theoretically this should provide the base model for curing most illness by a frequency entrainment derivative. The two hundred forty six cells may represent the basis set for the human body behavior.

As such like the piano with 88 keys we should be able to derive all combinations of frequencies of these units by assigning these a class of 200 body parts with their commensurate frequencies.

Piano Analog to Optimized Intelligent Expert Systems

Diagnostic Expert Based on 200 Body Part Reuse

If you know the frequency of 88 piano keys then you know the behavior of all 2̂88 keys. You know all combinatorial instances of keys and their corresponding frequency.

Having knowledge of their frequencies provides the information to rectify the aberrant signals.

If these 88 (200) keys are a basis set of objects theoretically this should provide the base model for curing most illness by a frequency entrainment derivative. The two hundred forty six cells may represent the basis set for the human body behavior. As such like the piano with 88 keys we should be able to derive all combinations of frequencies of these units by assigning these a class of 200 body parts with their commensurate frequencies.

Pattern Recognition Text
A piano keyboard is a pattern recognizer. We can consider the 88 unpressed keys as an analogy to human homeostasis or the Original start state of a sound inducing process of an instrument with 2̂88 patterns
When several keys are pressed they produce a set of audible Frequencies or an analogous set of medical symptoms.
The goal of the diagnostic process is to collect those frequencies (Symptoms) and restore them to the un pressed positions. This functionality is captured in a .Net application with a 100 bit body model This computes the set complement of a set of symptoms
The graph shows the routing or leveraging of history in a diagnosis. Above (111) routed to 110 via 0.5

International Medical Diagnosis (Cure Strategy)
Execute a Search based on Piece removal or process of elimination to find the collection of conditions responsible for system aberrant behavior. Note Checkmate in the game of chess is in part based on a search for a collection of conditions which depict a king being checkmated. The piece removal strategy is also the key to reducing an intractable problem space to a point that a less than Herculean effort could be launch to find or checkmate a king.
Design of the International Medical Diagnosis Expert System is based on a frequency resolution strategy. Each component in the body has a unique frequency, based on this must provide feedback to restore the component back to its specified frequency by an oscillator or commensurate technology. A scanning frequency is emitted until the troubled area responds accordingly.
Nano technology providing a feedback test and potential cure strategy. Nano blood tester provides a variety of test options and in the not to distant future could operated internally on condition which currently require intrusive entry.
Genome Mapping will render a road map of a number of medical conditions. These genome tools couple with a complete medical history of an individual could provide very powerful knowledge bases of illness eradications.
Populating a World Wide Internet Knowledge system by region will provide a roadmap for solving illness problems by example

International Medical Expert System
Leverage Past History within Seconds on PDA.
Answers the Question the Customer Asks.
Ask relevant questions about Current Ailment

Military Fighter Jet Game Combat and Defense
Using Frequency Games Model
Many frequency channels will allow playing multiple combat targets.
Virtually no think time between moves in defense since they are optimal.
Excellent strategy against multiple attacks. Each Boolean collection is an autonomous model of a Chess board configuration that can be played in parallel.
When implemented in a mini-max game context exploiting frequency driven tracking tactics optimal defensive and offensive tactic. Note by definition dogs fights exploit optimal path games theory.

Expert Systems
Chess games architecture
Enterprise Resource Planning
Enterprise Trouble Shoot System

Fighter Jet Games Model

Games Architecture

List of All patterns and Past history depicts optimized or games architecture. Each node in this architecture for the most part has a parent and a set of children relationship. This provides a forum to engage in an interactive game. Please see development of game strategy in chess game when a list of player work their way down the game lattice capturing the opponents piece after each move. Pg 57 In addition to this capture strategy for scoring. Past history of previous game are used to determine the best routing when faced with ambiguous decisions. This type of routing is also used in leveraging medical past history which may establish a propensity to routing to a specific state based on prior illness patterns.

Nano Control Structures
Nano control structures are entities which have characteristics which provides control for Nano objects. As a specification, is a collection of processes or process models which provides instructions to a Nano regime of objects based on leveraging past history (system visit count). Nano control could provide a catalyst for a set of reactions among regime members.
Many of the machine learning techniques and system optimization heuristics could be assembled to provide an overall strategy for an intelligent distributed objects infrastructure that could provide feedback for human body wellness.
The International Medical Expert Diagnosis provides diagnostic tools to help reverse body maladies however a sophisticated feedback and monitoring system must be incorporated in order to accurately assess the state of the body and provide this information to the Oracle.
Intelligent Nano control structures are objects that have a set of behaviors and are capable of learning new behaviors subject to the environment.
It is judge at a very sophisticated deployment strategy; these intelligent Nano control objects will be able to facilitate internal and external surgeries like skin graphs and brain tumors.
The medical profession will be greatly enhanced by the marriage of Nano control structures and Optimized Intelligent Search Technology. These innovations will provide a panacea for many types of inoperable tumors and delicate medical maneuvers which are impossible by today's standards.
Frequency control theory and the corresponding spectral frequency of objects will provide the fundamental communication strategy among objects.
Tumor detection, cancer treatment, repair and feedback mechanism for damaged body tissue, signals routing to damage organs.
Example of Nano control technology: Photosynthesis, laser eye surgery, microwave communications, cancer radiation treatment etc.

Explanation of the Chess Space Search Problem

The chess game is the classic example of an intractable search problem which was achievable based on removing pieces from the game board to eliminate a seemly inexhaustible pattern space. Piece remove appears to be a very robust and effective way of reducing pattern space and is central to the search, leveraging past history and processing power defined herein.

The intractable large search space was used to model very complex systems in medicine, business, and information science and has proven to provide unparalleled search, memory of history and processing power primarily in the context of expert system and software robot design.

The capture states of a chess game was utilized to leverage a finite set of basis vectors which was removed by defining medical symptoms or removed to satisfy a search heuristic (process of elimination). The capture states in chess actually embody the behavior of the game since the piece moves to capture are trivial and are too intractable to remember. In the context of achieving capture states we can remember past episode and leverage this past history in seconds.

Processes Summary
Thin client Web enabled Medical Expert System integrating symptom, medical questions, feedback, diagnosis, and treatment plan.
Next Move algorithm leverages past history to provide most appropriate decision as in the case of medical diagnosis.
Diagnose illness from very large knowledge base of similar cases by a (classification & clustering) technique.
Extremely Scaleable Classification Strategy via Medical Oracle tool leveraging previous diagnosis
Example of Expert System Problem Solver Models
Process and Optimization mathematical Modeling tool
Biological Transfer Function (Cure Path for New Diseases) tool
Collaborative approach to Genome Research and software tool
Collaborative approach to Bio-terrorism and Research tool

Intelligent Search
Set Complement Process for information retrieval facilitating exceptional processing power and information retrieval performance for primarily expert system applications.

Leveraging Past History for Tactical Decisions

High Speed Communications
Piece Reduction Algorithm from Chess (eliminates intractable no. of patterns in Chess)
Use of Basis Vectors to determine domain search scope (analog: piano 88 keys basis vectors or x, y, y in 3D). Technique defines a searchable problem space.
Provides a data structure that can facilitate the minimum path for a collection of discrete objects.
Accommodates a search engine capable of leveraging past history within seconds.
Unparallel pattern processing strategy based on object removal from a collection constituting a search space.
Optimal Intelligent Search (OIS) provides the potential for Millions of bits per second throughput and is greatly facilitated using the OIS architecture. It uses a linear search strategy by selecting the highest frequency at each level.
The model is similar to two chess players making millions of decisions or moves each second. OIS exploits the search heuristic of a graph by traversing the height of the graph or levels, and establishing decision points and visit count as a means of leveraging past history.
It is judged that this technology is a key way to empower a system to learn and thereby exhibit intelligent behavior.
OIS facilitates war dog fighter jet games applications since it is modeled as optimal path games theory model. In my opinion it is the optimal offensive-defensive strategy based on the optimal chess model described in the forthcoming summary. A single fighter jet in principle could defend against multiple concurrent enemy fire since each jet would playing an autonomous game against the enemy.
The ability to leverage past history, in addition to satisfying request subject to urgent time constraints (several seconds), is not only the mandate of critical care patients, wartime applications, and Business Enterprise reconciliation, but fundamental in defining criteria for New Generation Expert Systems. The forgoing provides a model based on the 4 billion capture states in chess, whose patterns can be easily be remembered by adapting OIS architecture. Notice the 4 billion states in chess represent actual decision points and the other 10̂10 are just paths to making the decisions.
We only need to keep track of the decision points for learning, remembering decades of information and optimizing the system. Notice we are able to define the present collection of medical history (based on body parts—medical records) and leverage system visit count accommodating all past visits to a pre-defined set of states. Past Medical decision points and present medical symptom defines the inputs to the diagnosis process.

Medical Expert Diagnostics Data Flow Data flow diagram shows piano analogy of medical expert system design. Specifically the instrument has 200 keys or frequencies which map to 200 body parts. The body communication key communications conduit is frequency since each body component has a unique frequency.

User Steps;
User experiences symptoms from some unknown medical condition
User takes his PDA device and presses medical history which provides several decades of past medical history.
User accesses 200 body component menu and carefully select the components that have associated symptoms.
As the user selects symptoms the system questions the participant with other likely

Components from the body system that may have contributed to the illness. These questions originate from past medical history instantiated by system visit count or the propensity to go in medical system states based on past behavior. System calibrates based on the symptoms selected and provides a diagnosis.

Treatment
System leverages frequency information obtained during the diagnosis phase and uses ultra-sound as a means of correcting or entraining aberrant frequency readings.
Oscillator vibrates until weak symptom signal from the body component is corrected
Record date of each visit count for leveraging past history
Oscillator Scanner—Bad frequency corrected by entrainment

Expert Systems Implementation on a PDA

Specifically we are analyzing a business object which will be offered as an internet service internationally. The key aspect of this analysis is simplicity and scalability of medical services offerings to a multi-lingual international community. Our target platform, which is undergoing rigorous analysis, is the Business phone which serves as an internet services conduit to a number of diversified markets. PDA requirements are that it can read a web page and support configuration to support a menu system of 200 human components complementing a diagnostic system. Each of the 200 human body components are either pristine or in symptom state.

A Medical Diagnostic system of good caliber is ubiquitous especially one that leverages past medical history of patient for present diagnosis in a few seconds. An Expert System caliber shell device on a PDA is also ubiquitous since it accommodates a number of different expert system applications on the same interface.

It is reasonable to project an optimistic sales forecast for the aforementioned expert system technology since it is not mainstreamed and pivotal modules leveraging past history, unparalleled search and processing power are being presented to our technology forum. The pattern search strategy appears to be many times more powerful than most if not all of the conventional search strategies that are currently mainstreamed.

Low priced expert systems of this caliber will also make it affordable and easily incorporated in a number of different languages and collaboration settings. Artificial Intelligence is underscored by search (intelligent) and search engine technology will probably dominate many computer driven initiatives, hence the search technology leveraged in this endeavor will be the commodity which redefines the foregoing expert system implementation.

Electronic Medical Record Text

Electronic Medical Data provide a layer of urgent response capability affording medical health care professional to quickly access victim's medical history so life saving decisions can made in an expedient manner.

Optimized Intelligent Search technology provides for a minimal interface to embody this information fundamentally presenting a list of 200 Medical metrics and their commensurate medical history. As professionals are query the PDA or cell phone technology they are arise of the propensity of previous conditions, surgeries etc.

Leveraging past medical history in seconds is the key innovation here not only render a much more accurate routing to illness states based on medical record but facilitating these result to be available in seconds.

The electronic medical record in conjunction with the International Medical Expert Diagnosis system will provide the urgent medical response strategy to prove an improved chance of survival.

Frequency Analysis of the Human Body Text

The model of the Human Body as a Frequency Spectrum captures the Diagnosis and Treatment strategy of the International Medical Expert Diagnostic System. Each body part has a unique frequency which we can leverage in deriving a heuristic to examine the body under illness conditions.

The system provides and interface which list the body components and their commensurate frequencies on a PDA.

The customer uses his hand held device to access and menu system with 200 body components.

The user selects the body component the he is experiencing a symptom and the system keeps track of all symptoms selected.

As symptoms are collected the system will provide a list of additional question based on leveraging information from past history.

When all symptom are inputted the user presses the diagnosis button and the symptom provides a frequency collection (known behavior) or diagnosis.

Treatment

System uses ultrasound interface to entrain aberrant frequency in conjunction with other frequency base therapeutic remedies.

Diagnosis and Waveform Behavior

Human Body is a map of a frequency spectrum therefore analyzing this system requires frequency based tools. Body parts correspond for the most part for all humans. Consider a body part as corresponding to a location and a behavior. When doctors examine patient responses usually involve various body parts are not well. Since body part denotes location and behavior, frequency provides an excellent feedback as to the state of the body part. Frequency denotes location and behavior of each respective body parts. The behavior of combinations of symptoms on body parts resembles the collection of corresponding frequencies and their commensurate wave form behavior.

http://www.spiritual-healer.com/rife-faq.htm

Rife Machine

Rife equipment is an instrument that generates specific user-inputed frequencies and harmonics based on Rifle's observations that intensifying the resonant frequency of a specific virus or bacteria destroys it. These frequencies and harmonics are transmitted to the body via hand-held, footplate, or stick-on electrodes.

How does a Rife Device work? Every molecule vibrates, or oscillates, at it's own unique frequency and resonates with that frequency. The living microbes {bacteria and viruses} Rife studied are invisible to the human eye because they are in the ultraviolet spectrum and don't reflect light. So, Rife, using the resonant frequency principle, devised a way to focus light into wavelengths that resonated with the signature frequency of the viruses and bacteria he was studying, “which illuminates these virus in their own characteristic chemical colors by emission of coordinative light frequency” making the virus “readily identifiable by the colors revealed”. These viruses and bacteria are so small that they cannot be seen with an ordinary microscope: “8000 to 17000× magnification is sufficient to see them”. Rife's invented and built his Universal Microscope to see and study these microbes in their living environment. Today's modern electron microscopes cannot even duplicate this feat.
International Medical Diagnostic (IMED) interface with a Diagnosis treatment and Cure Strategy. IMED operates as an intelligent router which records the state space or past history of a system then leverages this history and behavior parameters of the system. When the diagnostic system has aberrations, past medical history is incorporated to obtain a relevant diagnosis. The storage strategy of this method will dynamically classify and provide an extremely fast retrieval method for collections of body components.
Frequency Entrainment is one of the key concepts which provide a process to correct aberrant frequencies in a nature and scalable fashion. When there is a noisy signal a strong signal could entrain or over power the weaker sign thereby restoring the system back to good operating condition.
Use of set theory and storing set heuristics will cause the system to learn to efficiently navigate probable cause of a disease. Use of past medical history should enhance and promote successful questioning and response strategy. Boolean questions and search architecture facilitates efficient question strategy and coverage of many possible aberrant states.
Medical Treatment will be accomplished by identifying the relevant frequencies from the body components in the Diagnosis phase and using these frequencies to entrain or correct aberrant frequencies.

Linear and Non Linear Progression of Illness and Past History

The ability to capture linear and nonlinear system behavior has been the hallmark of system analysis. The proper behavior of any system can only be capture if there is a clear depiction of the state activity and progress of state transients. In deriving methods to effectively capture past history we observed there are at least 2 different behaviors as systems evolve through their states. Linear system are predictable and can be quantified and model using a number of methods. Non-linear systems has always been problematic and have given rise to fields of study like neural networks which attempt to learn the non linear behavior of these systems.

Using a graph approach we attempt to capture non-linear behavior the show a non-linear processing of events. As opposed to removing one object at each level, several objects may be removed at a level to depict actual non linear activity observed in the system.

It is considered that both a linear and non-linear design pattern be used to represent in disease and illness progression.

International Medical Expert Diagnosis

Menu

Scaling Factor 20-1000 objects

Summary and Set Complement

List Basis Body Components and Frequencies

Frequency Medical Analysis

Illness Classification

Diagnosis

Treatment Information

Language Preference

Medical Processes and Procedures

Question about Body Components

Each body component form a collection of 200 metrics which are evaluated as being OK or Not. This is the fundamental question that is driven through each component in aberrant body systems. Past medical history is leveraged to indicate the most likely state that a body would be evolving towards during an illness crisis.

Medical Processes

Medical processes and procedures are stored of each body component and fundamentally a strategy to address combinations of different system is addressed by modeling the body as a collection of frequencies which are mapped to symptoms. This is analogous to each note on the piano being mapped to a specific musical frequency.

Body Component Images

System has the capacity to store a large number of medical systems which correspond to the large number of body components. Images are capable of being resized to facilitate different aspects of analysis.

Medical Prescriptions

Fundamentally the system was design to exploit frequency entrainment or leveraging frequencies discovered during the diagnostic phase and attempting to rectify aberrant frequencies with ultrasound.

Set Complement and Missing Information
Set Complement was used in Medical application in conjunction with a symptom profile. The Complement Process represented the restoring signal along with a symptom profile to have no symptoms. E.g.(11111111111 . . . 1)
The complement process appears to be a model for human memory. Based on partial information the complement provides the rest of the set of objects.

Capture States and Basis Vectors

The game of captures states or optimal games could provide a frame work for optimizing resources. In the game of chess there are 4 billion optimal paths or games. As the game of capture states progresses, the one who move first should win if played properly since the weights and positions are linear.

The architecture provide for process optimization strategies and minimum path from a collection of resource subject to constraints.

The lattice of capture states provides a hierarchical database which breaks of objects in classes so they can be search for very efficiently.

The analysis of the human genome is considered an ideal candidate for the lattice of capture states since the classification structure naturally ranks it objects in terms of behavior.

Target Deliverable

Software Robot for Health Care Diagnostics
Mathematical Models
Basis Set Collections
Input Process Output Function Format
Intelligent Agent Patterns
Set Complement
Factoring Large Prime Numbers Multiplied

1. A method which leverages Personal Digital Assistant (PDA), internet and telecommunications technology infrastructure as a platform for decision support and case evaluation of a problem over a period of time, comprising of the steps of:
Inputting and processing irregular system data using a PDA device;
Configuring a knowledge base by selecting the appropriate set of entities corresponding to system errors as the knowledge base comprises a plurality of components which are evaluated as pass or fail, date of incident field and an additional metric called system visit count recording the number of times a component had a symptom or system incident.
Storing the knowledge base on the PDA leveraging decades of past history for proper assessment of present conditions;
Analysis of knowledge base metrics for proper system function is primary viewed as satisfying a search driven heuristic as there is partial information available which is incrementally updated by system questions and leveraging information from past history, a set of conditions match and collection of knowledge base basis objects which are mapped to frequency reading used as strategy to define system aberrations figure pp 7, 23, 24

2. A method leveraging Personal Digital Assistant (PDA), internet and telecommunications technology infrastructure as a platform for decision support and medical evaluation of a patent over a period of time,
Inputting and processing symptoms from patients using a PDA device;
Configuring a knowledge base by selecting the appropriate set of symptom corresponding to the patient's ailment as the knowledge base comprises a plurality of body components which are evaluated as pass or fail, date of incident field and an additional metric called system visit count recording the number of times a component had a symptom or system incident.
Storing the knowledge base on the PDA leveraging decades of past medical history for proper assessment of present conditions;
Analysis of knowledge base metrics for body wellness is primary viewed as satisfying a search driven heuristic as there is partial information available which is incrementally updated by system questions and leveraging information from past history

3. A method defined in claim 1 providing a menu of body components on a PDA comprising a basis set of vectors of a specified problem domain, leveraging finite set design and reuse of system components, with system components evaluated as being acceptable or unacceptable, leveraging past history to determine the routing or next probable state based on collecting system feedback metrics. PDA has capability to compare statistics taken from globally derived profiles, since system uses body component frequency from MRI and other waveform technologies, since diagnosis is in the form of a power set of frequencies diagnosis spans an intractable number of treatment strategies using frequency entrainment or other frequency based measures, may be derivable addressing issues of bioterrorism. Frequency diagnosis and treatment strategy could greatly expand medical market overseas in markets where we would not have access, etc. (See figure) p 23, 24, 25

4. A method that defining an operating environment providing for expert systems services and advice based on exploiting minimum path and process technology defining an unparalleled search process, accomplish by removal of a piece from the game domain thereby also elimination all pattern associations with the said piece on the average removing an intractable no. of pattern, i.e. chess, FIG. 22 processing and leveraging of past history in seconds to accomplish an intelligent demeanor answering medical request and advice, in addition to mainstreaming health care internationally by addressing access and availability paradigm via PDA for health care and other service delivery mechanisms, hence facilitating close to minimum circuit and program code implementation accommodating small size of PDA by optimizing a collection of object per minimum path

5. A method of establishing optimized processes for Intelligent search by establishing a expert domain of basis vectors or objects whose linear combination spans a vector space as in the case of a piano, 88 keys span music orchestration space, as the said vectors are mapped to typically 200-1000 body components, as this ensemble comprises a list of objects which will be used to select various body symptoms or aberrations, as these components or vector undergo system cycles visit counts are record to define the propensity of a component to be visited based on prior logging of medical condition etc, as the frequency reading of the symptom component are record the commensurate frequency reading of body components are recorded for further processing and Diagnosis is provided after system questions and symptoms are documented.

6. A method to establish system treatment based on frequency related data from the diagnosis phase of IMED, as an MRI frequency chart of a human body components are used to establish the proper frequency level that may be used to design an ultrasound treatment plan. Knowing 200 frequency level actually defines 2̂200 frequency levels and their known behaviors per combination expansion, as specifying frequency entrainment of aberrant signal should be definitive by knowing the frequency expansion of the basis vectors.

7. A method of defining a knowledge base comprised of a collection of body components along with their commensurate body systems, a series of system centric question derived from a summary of body components and corresponding body system, enumerated based on system visit count, reducing pattern space, as out of scope questions are retired. A medical method including the following fields: Date, system visit count, body part, body system, body part state, complement, diagnosis, diagnosis leveraging past history, date of illness, method defining an anatomical system with cause of the problem, storing the consultation history database or electronic data record in a computer. See figure

8. A method leveraging set and set complement structures to define acceptable system states and aberrant system components, facilitating a strategy to simulate system acceptability and analyze corrective body states and symptoms on body components, facilitating a strategy to simulate body wellness states and analyze corrective frequency driven measures, using a set complement model which monitors the collection of state conditions deviating from the norm, using user input as a means of recording symptoms defining a tool which capture a collection of metrics or body component defining acceptable or unacceptable, 1 or 0 etc. (See figure) p 38,39

9. Body Component symptom interface and diagnosis module;
User will enter user-id and medical past history will be displayed
The system will ask the user to select the body components with symptoms from menu of 200 body components
System will store the symptom on the body component for later processing by input 0.
The system will inform the users of potentially different kinds of symptoms, system will provide additional question based on medical history to extract additional symptom information, questions are derived based on body component—body system construction filtered by component visit count
After all information (medical history and current symptoms are upload the system will provide a diagnosis.
Treatment
Frequency information is calibrated from the symptom—component frequency derived from an MRI component frequency table, data during the diagnosis phase and leveraged to develop a frequency driven input strategy to alleviate symptoms using a frequency treatment such as entrainment, the body can be viewed as a frequency spectrum considering body cells and body components therefore a frequency treatment approach to restoring full frequency oscillation harmony is adopted here .figure pp 22

10. The method define by medical process and procedures system recall based on graph architecture exploiting parent and child relationship fundamental scenario enumeration begins with a list of body component, the string 01011110111 or any other string has a specific set of children relationship serving as a strategy to store past history, each bit collection has a unique path which can be exploited to provide complex processes and procedures to specified path functions, medical recall of processes and procedures is enabled by establishing said basis vectors for the expert system and specifying the corresponding frequency for the body components, herein all diagnosis of 2̂200 diagnosis scenarios and their commensurate treatment scenarios are derivable from this relationships since the finite collection defines explicitly the behavior of the entire domain space, every parent has a unique set of child paths which are totally See pp 21

11. A method defining set complement which identifies where information is located and provides the strategy to restore collection of missing data back to its original complete state, determines all missing data from the target set or basis set of objects, it is a set operation which keeps track of missing information and object location, in performing diagnosis or system analysis on a system we assume the initial state of homeostasis defines a well working collection of components, as system inconsistencies and flaws arise, the diagnosis process keeps track of all the original start states of the system, collects all the system aberrations or symptoms and identifies the set of objects which needs to be restored to make the system complete. The forte of the diagnosis process is the ability of keeping track of trillions of trillion of system states leveraging a search process that is fundamentally derived from the chess piece capture process. See figure pp 8, 38, 39

12. A method of analysis leveraging Optimized Intelligent Search via pattern reduction of the search domain through piece removal, search heuristic using piece removal an graph traversal strategy, leveraging decades of past history using a basis set of vectors spanning the problem domain, 88 piano keys, applying visit count to basis system to capture past history, implementing graph heuristic to optimize the processing of a collection of objects. FIGS. 40, 50, 52, accessing medical history in seconds is achieved by using a finite basis set which captures all states of a system in addition to recording system visit count which provides the necessary routing of past history to new medical conditions p 17, 19.

13. A medical record accessible from an electronic delivery PDA system providing patient's easy access at a medical scene as system will be capable of leveraging many years of past history within a few seconds since the computer program data structure labels each component with a metric recording system visit count, as the time counter groups a collection of symptoms that occurred during the same period of time.

14. A method of obtaining feedback initially driven from patient observation assigning symptoms in medical software, medical support for calculating patient baseline and vital signs summary, mapping to illness or scheduled examination, use of blood, urine and vital signs analyzers facilitating a feedback strategy. The method defined instantiates intelligent behavior by the ability to leverage past history to make present decisions, apparently the survival of all intelligent things depends on the ability to remember past events within seconds and learn See figure pp 17, 25
See figure pp 32

15. A method facilitating optimized intelligent search leveraging the search power of base 2 which can find any pattern in the 10̂20 search space˜2̂400 in 400 finite steps as approach provides the optimal path to each point or set of systems in the search domain establishing a reusable pattern space for system design, identifies frequency of body components from MRI by listing the body components with symptoms, using these groups of symptoms as a mechanism to provide a diagnosis and ultimately a treatment plan. figure pp 51 piece removal (chess) process or symptom identification strategy to gather the appropriate symptom defining the diagnosis, system visit count assists in identifying the correct routing parameters to diagnosis conditions, identifying chronic conditions could change overall diagnosis as piece removal process appears to be an unparallel process for reducing pattern space and has numerous applications to locate objects in a domain space, as removing an object divides the search space in half, as IPV6 application could easily leverage piece removal as a search heuristic. See figure pp 11, 50,53

16. A method of defining a Mathematical forum to model Critical Care operations settings or Jet fighter pilot game strategy, providing state space of human body component interactive processes based on the fact body components are a collections of various component systems, Fighter Jet Games architecture leverages all patterns and past history of combat scenarios, depicts optimized or games strategic moves, each node in this architecture has a parent and a set of children relationship, as this provides a forum to engage in an interactive fighter game. or a critical care analog of combating a life threatening condition, as the chess war strategy models many interactive game heuristic and should adapt well to the objectives of a critical care setting leveraging real-time past history, search and processing power to routing a state based on prior illness patterns. See figure pp 22, 45, 49

17. The method defined Classification: will instantaneously classify objects that are 100 bits long however there is no practical limit to the length of the bit string to be evaluated. The Classification model is a collection of basis vectors that define human body parts. The appropriate symptoms are identified by changing a 1 to a zero on the affected body part. See figure pp 49, 51

18. A method providing for leveraging 200 body components facilitating 2̂200 symptom patterns, mapped to 200 known frequencies provides for a known pattern space of 2̂200 since leveraging a linear combination of 200 know base frequencies, Leverage Past history in seconds since as a collection of frequencies are selected their corresponding system visit count to the registered states are available. See figure pp 22 PowerPoint

19. Specification of an Expert System unparalleled search, leveraging past history, processing power, use of basis vectors to establish search space, define basis collection of objects to address search space, map collection of components to unit vectors. Select symptoms based on body components listed. Interact which question from component-system module to identify the most probable cause after all questions are answers and symptoms listed select diagnosis key see pp 7,8,12

20. Leveraging Memory for critical care procedures, processes and treatment strategy, diagnosis leverages collection of frequencies, facilitating a scalable frequency based treatment approach e.g. 2̂200 combinations, automation of next step procedures based on feedback from International Medical Expert Diagnosis, string have optimal pattern children from graph enumeration used as a tool to respond dynamically to changing states in the body condition.

21. A method of providing System Problem solver services based on defining a collection of basis vectors assigned a plurality of objects, identifying inconsistencies or fault on said objects, tracking system visit count on objects, using object piece removal to reduce pattern space, ultimately arriving at a plausible cause based on object original configuration, set complement determines all missing data from the target set or basis set of objects, it is a set operation which keeps track of missing information and object location, in performing diagnosis or system analysis on a system we assume the initial state of equilibrium defines a well working collection of components as system inconsistencies and flaws arise, the complement process keeps track of all the original start states of the system, collects all the system aberrations identifies the set of objects which needs to be restored to make the system properly functional the forte of the complement process is the ability of keeping track of trillions of trillion of system states leveraging a search process that is fundamentally derived from the system visit count and piece capture process.

22. A method defining identifying expert system frame work for applications leveraging unparalleled search using piece removal i.e. capture chess diminishing pattern space, leveraging past history using a reusable set of basis vector spanning the target domain space tracking each object by system visit count and leveraging graph search heuristic for object classification, pattern processing and search location, with process for utilizing IPV06 network resources by leveraging object piece removal, leveraging of past history and strategy recall of operating events, expert framework uses different basis vectors but reuse the same search strategy and pass history process for routing search efforts, as interchangeability facilitates using the same expert shell to accomplish fulfill different application requirements. Figure pp 69,70,71,72, 73 which characteristics as follows:
Next Move Process A decision enumerating process which incorporates all past history and provides the optimal decision or ordered set of decisions with a very time sensitive strategy, example: “based on all available past history make the best tactical decision in a critical operating procedure next move seems to provide the base case for interactive intelligent communication since it uses a collection of basis vectors with their corresponding visit count to define past history,
Frequency Based model: The OIS architecture provides the potential for optimal path routing of collection of objects, each collection of objects in a graph represents a different board configuration which actually represents simulated games played in a massively parallel strategy. The opponent can commence playing the games in a mini-max game strategy. The frequency based model for homeostasis leverages body component frequency integrity as a diagnosis and treatment strategy,
Scalability list or collection of bit vectors facilitating the expression and enumeration of large strings of objects. The OIS chess game architecture was based on a list of 32 unit vectors, the power set or list of all possible combinations of the 32 object results in 4 billion states, these 4 billion states represent the critical path of the traditional chess games which has 10̂120 states the vast majority or 10̂110 states are Markovian or routes to capture states. For the analysis of the game of optimal paths these Markovian states can be neglected, the list of unit vectors is part of the search strategy, compression strategy, flexible architecture and reusable system tool.
Expert System: Problem Solver is an intelligent agent with a powerful architecture accommodating stealth search, object classification, object reuse, memory of past scenarios, extremely scalable and extendable communications, since objects and components are fundamentally collections, OIS defines it architecture in terms of an ordered collection of Basis vectors that span the target business model, applying a piece reduction, set complement or lattice search algorithm problems involving state space can be simulated and solved.
Operational Speed: Game playing speed between OIS players could be in the 10s of Megahertz range based on the optimal path architecture there is a drastic pattern space reduction, this critical path is searched using graph search techniques; piece reduction and set complement approaches think time for search is drastically reduced since optimized chess is a linear model.
Classification: will instantaneously classify objects that are 100 bits long however there is no practical limit to the length of the bit string to be evaluated the Classification model is a collection of basis vectors that define human body parts. The appropriate symptoms are identified by changing a 1 to a zero on the affected body part, this ordered collection of symptoms and unaffected body parts form a search string in a database of other body symptom profiles, as new profiles are added to the database the form clusters of profiles with similar traits these cluster form a solution strategy for similar collection of bits.
Pattern Recognition: will recognize virtually any string including the past game patterns on a trained lattice, including all appropriate visit counts to systems, evaluation is the length of the string e.g. 256 steps since the architecture processes only the optimal path of a process, this actually provides memory of past events.
Memory of Past Events The OIS architecture provides for remembering past scenarios or games since the traversal of the game graph takes the optimal path at system configuration each string has a unique path that it traverses visit count will ultimately route a string leveraging past medical history.
Reuse of systems: fundamental design exploits the characteristic of a musical instrument, specifically a set of basis notes which are reused as songs are created.
Search: There are two powerful search strategies used in the Medical Oracle for diagnosing patient conditions the complement of a set is used to determine the appropriate electric response to restoring a body signal pattern to the desired levels the piece reduction process is an elimination approach of discovering an aberrant collection and ideally a cause.
Complement Process: takes a string representing an aberrant system behavior and establishes the restoring string profile integrating past history.
Piece Reduction Process: a search process derived from chess capture rules, piece reduction affords a reduction of the enormous search space of chess and makes it feasible for the game to shrink to a size that checkmate can be realized each piece that is remove from chess eliminates thousands of trillions of relationships.
Interchangeability: a design which allows different solutions to share the same GUI, processes, or architecture
Intelligent Agent: application development environment to design and implement specialized search engines to acquire and process resources.
Learning Agent: two players in OIS chess games can learn in the length of a bit profile string (100 bits) at speeds in the Megahertz level. (loan agent,
Troubleshooting agent, business analysis agent, medical diagnosis agent)
OIS Search Lattice per search lattice FIG. 101 depicting objects storage, retrieval, search strategy, zero critical information loss—per visit count. on basis vectors this is analogous to remembering all capture states in a chess game (4 billion). The other 10̂110 Markov states are not important since they are not repeatable and doesn't represent a decision.
Data Compression using entropy or visit count and piece removal from the system FIG. 106. Estimated 50% data compression based on piece removal alone this is information loss however the information that is saved can re-establish information that can be learned
Intelligent behavior is the ability to Leverage past history to make present decisions it appears the survival of all intelligent things depends on the ability to find our way back to the goal state or learn. Losing the ability to leverage past history would be analogous to a person having a failure of the hippocampus rendering the creature helpless since it would have to learn everything all over again each day assuming there was some capacity for short term memory.
The Optimized Intelligent Architecture and all applications that use past history capture states to leverage making present decisions including the Medical Oracle medical expert system is the target of this process. All past history is not relevant here just as all moves in a chess game need not be remembered e.g. non reusable move the information that is considered past history is the decision points analogous to the capture states in a chess game.
